SEQUENCE LISTING (24 sequences)

SEQ ID NO: 1 - DNA, 55 bp, Artificial Sequence
  OligoAmp_pF1
ttgaaagtatttcgatttcttggctttatatatcttgtggaaaggacgaaacacc

SEQ ID NO: 2 - DNA, 54 bp, Artificial Sequence
  OligoAmp_pR1
tttcaagttgataacggactagccttaggttaacttgctatttctagctctaac

SEQ ID NO: 3 - DNA, 57 bp, Artificial Sequence
  OligoAmp_pR2
gagtaagctgaccgctgaagtacaagtggtagagtagagatctagttacgccaagct

SEQ ID NO: 4 - DNA, 97 bp, Artificial Sequence
  Insert fragment
cgtctctgttttagagctagaaatagcaagttaaaataaggctagtccgttatcaacttgaaaaagtggcaccgagtcggtgcttttttgggagacg

SEQ ID NO: 5 - DNA, 61 bp, Artificial Sequence
  NGS1st_stgRNA_pF1
acactctttccctacacgacgctcttccgatcttggctttatatatcttgtggaaaggacg

SEQ ID NO: 6 - DNA, 62 bp, Artificial Sequence
  NGS1st_stgRNA_pF2
acactctttccctacacgacgctcttccgatctatggctttatatatcttgtggaaaggacg

SEQ ID NO: 7 - DNA, 63 bp, Artificial Sequence
  NGS1st_stgRNA_pF3
acactctttccctacacgacgctcttccgatctcctggctttatatatcttgtggaaaggacg

SEQ ID NO: 8 - DNA, 65 bp, Artificial Sequence
  NGS1st_stgRNA_pR1
gtgactggagttcagacgtgtgctcttccgatctgccttaggttaacttgctatttctagctcta

SEQ ID NO: 9 - DNA, 66 bp, Artificial Sequence
  NGS1st_stgRNA_pR2
gtgactggagttcagacgtgtgctcttccgatcttgccttaggttaacttgctatttctagctcta

SEQ ID NO: 10 - DNA, 67 bp, Artificial Sequence
  NGS1st_stgRNA_pR3
gtgactggagttcagacgtgtgctcttccgatctatgccttaggttaacttgctatttctagctcta

SEQ ID NO: 11 - DNA, 62 bp, Artificial Sequence
  NGS1st_stgRNA_pF1r
gtgactggagttcagacgtgtgctcttccgatcttggctttatatatcttgtggaaaggacg

SEQ ID NO: 12 - DNA, 64 bp, Artificial Sequence
  NGS1st_stgRNA_pR1r
acactctttccctacacgacgctcttccgatctgccttaggttaacttgctatttctagctcta

SEQ ID NO: 13 - DNA, 56 bp, Artificial Sequence
  NGS1st_sgRNA_pF1
acactctttccctacacgacgctcttccgatctcttgaaaaagtggcaccgagtcg

SEQ ID NO: 14 - DNA, 57 bp, Artificial Sequence
  NGS1st_sgRNA_pF2
acactctttccctacacgacgctcttccgatcttcttgaaaaagtggcaccgagtcg

SEQ ID NO: 15 - DNA, 58 bp, Artificial Sequence
  NGS1st_sgRNA_pF3
acactctttccctacacgacgctcttccgatctcgcttgaaaaagtggcaccgagtcg

SEQ ID NO: 16 - DNA, 61 bp, Artificial Sequence
  NGS1st_sgRNA_pR1
gtgactggagttcagacgtgtgctcttccgatctttaagtcgagtaagctgaccgctgaag

SEQ ID NO: 17 - DNA, 62 bp, Artificial Sequence
  NGS1st_sgRNA_pR2
gtgactggagttcagacgtgtgctcttccgatctattaagtcgagtaagctgaccgctgaag

SEQ ID NO: 18 - DNA, 63 bp, Artificial Sequence
  NGS1st_sgRNA_pR3
gtgactggagttcagacgtgtgctcttccgatcttattaagtcgagtaagctgaccgctgaag

SEQ ID NO: 19 - DNA, 57 bp, Artificial Sequence
  NGS2nd_pF1
  misc_feature (30)..(37): n is a, c, g, or t
aatgatacggcgaccaccgagatctacacnnnnnnnnacactctttccctacacgac

SEQ ID NO: 20 - DNA, 53 bp, Artificial Sequence
  NGS2nd_pR1
  misc_feature (25)..(32): n is a, c, g, or t
caagcagaagacggcatacgagatnnnnnnnngtgactggagttcagacgtgt

SEQ ID NO: 21 - DNA, 29 bp, Artificial Sequence
  stgRNA_scaffold_pF1
gggttagagctagaaatagcaagttaacc

SEQ ID NO: 22 - DNA, 21 bp, Artificial Sequence
  stgRNA_scaffold_pR1
ccgactcggtgccactttttc

SEQ ID NO: 23 - DNA, 17 bp, Artificial Sequence
  U6_endogenous_ctrl_pF1
ctcgcttcggcagcaca

SEQ ID NO: 24 - DNA, 20 bp, Artificial Sequence
  U6_endogenous_ctrl_pR1
aacgcttcacgaatttgcgt

(19) United States
(12) Patent Application Publication
(10) Pub. No.: US 2022/0251634 A1
(43) Pub. Date: Aug. 11, 2022

(54) METHOD FOR RECORDING ELAPSED TIME IN DNA OF CELLS

(71) Applicant: INDUSTRY-ACADEMIC COOPERATION FOUNDATION, YONSEI UNIVERSITY, Seoul (KR)

(72) Inventors: Hyong Bum KIM, Seoul (KR); Ji Hye PARK, Seoul (KR)

(73) Assignee: INDUSTRY-ACADEMIC COOPERATION FOUNDATION, YONSEI UNIVERSITY, Seoul (KR)

(21) Appl. No.: 17/290,657

(22) PCT Filed: Nov. 12, 2019
(86) PCT No.: PCT/KR2019/015372

§ 371 Date: Apr. 30, 2021

(30) Foreign Application Priority Data
Nov. 15, 2018 (KR) 10-2018-0141093

(51) Int. Cl. C12Q 1/6827; G16B 30/20

(52) U.S. Cl. CPC C12Q 1/6827; G16B 30/20

(57) ABSTRACT

The present invention relates to a method for recording the passage of time in DNA of cells. More specifically, the present invention relates to a method for measuring time which has elapsed from a predetermined time point in cells using target genome editing system, and to a system for measuring time in cells. The method of the present invention is a new synthetic biological clock that enables the accurate in vivo measurement of the time which has elapsed from a defined time point to any time point. Through the system of the present invention, time information ranging from hours to weeks can be accurately recorded in vitro or in vivo in DNA of animal cells and living animals, and the time which has elapsed from a recorded time point can be measured at an unknown time point through DNA sequencing. Also, when the synthetic DNA clock of the present invention is used, it is possible to accurately record and measure the exposure time of cultured cells to chemicals and the lifespan of living animals remaining after time starts to be recorded in the living animals. In addition, temporal information regarding various intracellular signal transductions can be recorded and decoded in DNA in the cells using the synthetic DNA clock of the present invention.

### CROSS-REFERENCE TO RELATED APPLICATIONS

This application is a national stage of International Application No. PCT/KR2019/015372, filed Nov. 12, 2019, which claims the benefit of Korean Application No. 10-2018-0141093, filed Nov. 15, 2018, in the Korean Intellectual Property Office, the disclosures of which are incorporated herein by reference.

### SEQUENCE LISTING

The Sequence Listing submitted in text format (.txt) filed on Feb. 2, 2022, created on Feb. 2, 2022 and named SQL_.TXT (6,088 bytes in size), is incorporated herein by reference.

### TECHNICAL FIELD

The present invention relates to a method for recording elapsed time in DNA of cells, and more particularly, to a method for measuring time which has elapsed from a predetermined time point in cells using target genome editing system, and a system for measuring time in cells.

### BACKGROUND ART

In the most fields of science, it is very important to accurately measure time in the living body. In particular, it is very important to record and measure time in the field of biology because most of the biological phenomena are dynamic.

In the field of physics, the lapse of time has been measured using the decay of radioactive isotopes. This radiometric dating method depends on two principles: atoms of individual radioactive isotopes are converted into decay products at a constant rate; and all conversion reactions are independent of each other. Therefore, the number of the atoms of radioactive isotopes remaining in a certain substance decreases exponentially over time, and amounts of the radioactive isotopes and decay products in the substance may be measured to calculate the lapse of time from the half-lives of the radioactive isotopes. Such a dating method has been used to determine the ages of materials such as rocks or fossils.

In modern life sciences, electrical or mechanical methods are still used to measure time. However, a synthetic biological system capable of measuring a relatively long period of time, such as weeks, has still not been developed.

It is known that DNA serving as a genetic material is a medium that stores recent information. It is known that biological events such as chemical exposure, inflammatory response, signal transduction activity, and RNA transcription as well as the utility potential of certain metabolites are recorded in DNA using DNA-engineering tools such as a CRISPR-Cas nuclease system, and the like. However, little is known about the fact that temporal information such as elapsed exposure time to chemicals or lifespans of animals is accurately recorded in DNA in cells.

Although limited time information is recorded in a DNA sequence using methods such as site-specific recombinases, Cas1-Cas2-mediated oligonucleotide acquisitions, and base editing, such methods did not lead to a level of “DNA clock” because they have limitations on resolution and a recordable time range.

Accordingly, the present inventors have ardently endeavored to develop a method for accurately measuring time in living animal cells and animals, and found that the frequency of an intact target sequence decreases exponentially over time when indels are formed in target sequences in cells using a CRISPR-Cas9 system. Therefore, the present inventors have developed a synthetic biological system capable of accurately measuring units of time spanning from several hours to weeks by deriving an equation that represents the correlation between time and the indel frequency of the target sequence. Therefore, the present invention has been completed based on these facts.

### DISCLOSURE

### Technical Problem

The present invention is directed to providing a method for measuring time which has elapsed from a predetermined time point in cells, which includes: (a) transducing a composition for editing target genes into cells, followed by culturing of the cells; (b) harvesting some of the cultured cells at any time point (t) which has elapsed from a predetermined time point, followed by sequencing of target sequences from the genomic DNA of the cells; (c) measuring an indel frequency (IF) of the target sequence; and (d) calculating any time point using the following equation:

F=1−IF=e^{-λ(t-t}^{₀}⁾(t≥0,t₀≥0)

wherein F represents a relative frequency (ratio) of the copy number of an intact target sequence in the total copy number of the target sequence at any time point, IF represents an indel frequency of the target sequence measured at any time point, λ is a positive constant that represents an indel generation rate of the target sequence per unit time, and to is the latent time taken to express a transgene transduced into cells.

The present invention is also directed to providing a system for measuring time in cells, which includes an intracellular indel generation unit including a composition for editing target genes; an intracellular indel frequency measurement unit for sequencing of the target genes; and a time prediction unit for calculating the lapse of time at any time point from a predetermined time point using the measured indel frequency.

### Technical Solution

To solve the above problems, one aspect of the present invention provides a method and a system capable of accurately measuring any lapse of time in cells based on the fact that the frequency of an intact sequence decreases exponentially as in radiometric dating when an indel is generated in target sequences by means of a CRISPR/Cas9 system.

Another aspect of the present invention provides a method for measuring time which has elapsed from a predetermined time point in cells. Hereinafter, respective steps of the method will be described in detail.

The method for measuring time which has elapsed from a predetermined time point in cells according to the present invention includes [step (a)] transducing a composition for editing target genes into the cells, followed by culturing of the cells.

The composition for editing target genes according to the present invention may include a guide RNA, a target base sequence targeted by the guide RNA, and an RNA-guided nuclease.

In the present invention, the term “guide RNA” refers to an RNA specific to a target DNA, and the guide RNA may complementarily bind to a portion or all of the target sequence so that an RNA-guided nuclease can cleave a target sequence.

In general, the guide RNA refers to a dual RNA that includes two RNAs, that is, CRISPR RNA (crRNA) and trans-activating crRNA (tracrRNA) as constituents; or a type of RNA that includes a first section including a sequence complementary to some or all of a sequence in the target DNA and a second section including a sequence interacting with the RNA-guided nuclease. However, the guide RNA may fall within the scope of the present invention without limitation as long as the RNA-guided nuclease can have activity in the target sequence. For example, when the guide RNA is applied to Cpf1, the guide RNA may be crRNA. When the guide RNA is applied to Cas, especially, Cas9, the guide RNA may be in the form of a dual RNA including crRNA and tracrRNA as constituents, or in the form of a single-chain guide RNA (sgRNA) in which main portions of crRNA and tracrRNA are fused. The sgRNA may include a section having a sequence complementary to the sequence in the target DNA (which is named “a spacer region,” “a target DNA recognition sequence,” “a base pairing region,” and the like) and a hairpin structure for binding of Cas, especially, a Cas9 protein. More particularly, the sgRNA may include a section having a sequence complementary to some or all of the sequence in the target DNA, a hairpin structure for binding of Cas, especially, a Cas9 protein, and a terminator sequence. The above-described structures may be present in sequential order from 5′ to 3′. However, the present invention is not limited thereto and any type of guide RNA may also be used in the present invention as long as the guide RNA includes a main section of crRNA or a section complementary to some or all of the target DNA.

The guide RNA, particularly, crRNA or sgRNA, may include a sequence complementary to some or all of the sequence in the target DNA, and may include one or more additional nucleotides in an upstream region of crRNA or sgRNA, particularly, at the 5′-terminus of the sgRNA or crRNA. The additional nucleotides may be guanine (G), but the present invention is not limited thereto.

Also, the guide RNA may include a scaffold sequence that assists attachment of the RNA-guided nuclease.

In the present invention, the term “target base sequence” or “target sequence” refers to a base sequence that is expected to be targeted by the RNA-guided nuclease. In the present invention, the target base sequence also includes a desired sequence whose indel frequency is analyzed in the method of the present invention. In the present invention, because the guide RNA and the target sequence are present in the form of a pair in an oligonucleotide and a vector, each of which constitutes the oligonucleotide library and the vector library, the guide RNA present in one oligonucleotide or vector corresponds to the target sequence.

The term “target sequence” used in the present invention refers to a sequence that is used to analyze whether the activity of the RNA-guided nuclease caused by the guide RNA present in the form of a pair functions. That is, this may be determined in the step of designing or preparing each oligonucleotide, which constitutes the oligonucleotide library of the present invention, by the practitioners. Therefore, the practitioners may select a sequence expected to have target activity for a paired guide RNA or a sequence expected to have an off-target activity for the paired guide RNA and design the sequence as a target sequence in the designing step, depending on the purpose of implementation. The target sequence may include a protospacer-adjacent motif (PAM) sequence recognized by the RNA-guided nuclease, but the present invention is not limited thereto.

In the present invention, the guide RNA and the target base sequence targeted by the guide RNA may be a self-targeting guide RNA (stgRNA).

In the present invention, the term “self-targeting guide RNA” or “stgRNA” is an RNA that includes both of a guide RNA sequence and a target sequence in one nucleic acid sequence, and is simplified compared to a conventional CRISPR system in which a target sequence and a guide RNA binding complementarily to the target sequence should be separately designed. The stgRNA is characterized by reduced activity as compared to conventional guide RNA, and thus an indel frequency and activity can be measured over a long period of time. In one embodiment of the present invention, the stgRNA sequence was used to further simplify analysis of indels in the desired sequence and determine a possibility of measuring time for a relatively long period of time.

In the present invention, the term “RNA-guided nuclease” refers to a nuclease that may recognize and cleave a certain site on a desired genome, particularly, a nuclease that has target specificity for the guide RNA. The RNA-guided nuclease may include a CRISPR-associated protein 9 (Cas9) protein or CRISPR-associated endonuclease in Prevotella and Francisella 1 (Cpf1), both of which are derived from CRISPR that is a microbial immune system, or a nuclease whose activity is induced by chemicals, but the present invention is not limited thereto.

The RNA-guided nuclease may recognize a certain base sequence in the genome of animal and plant cells (including human cells) to cause a double strand break (DSB), and may form a nick (nickase activity). The double strand break may include all types of blunt ends or cohesive ends formed by cleaving the double helix of DNA. The DSB is efficiently repaired by means of a homologous recombination or non-homologous end-joining (NHEJ) mechanism in cells. In this process, a researcher may introduce a desired mutation into a target site. The RNA-guided nuclease may be an artificial or engineered, non-naturally occurring nuclease.

In the present invention, the term “Cas protein” or “Cas9 protein” is a major protein component in a CRISPR/Cas system, and refers to a protein that may serve as an activated endonuclease or nickase. The Cas protein may form a complex with crRNA (CRISPR RNA) and tracrRNA (trans-activating crRNA) to exhibit the activity thereof.

Information on the Cas protein or gene may be obtained from the known databases such National Center for Biotechnology Information (NCBI) GenBank. In particular, the Cas protein may be a Cas9 protein. Also, the Cas protein may be a Cas protein derived from the genera Streptococcus, Neisseria, Pasteurella, Francisella, and Campylobacter. Specifically, the Cas protein may be a Cas9 protein derived from Streptococcus pyogenes. However, the present invention is not limited to the above-described examples as long as the Cas protein has the above-described RNA-guided nuclease activity. In the present invention, the Cas protein may be a recombinant protein.

In the present invention, the term “Cpf1” or “Cpf1 protein” is a nuclease in a new CRISPR system that is distinct from the CRISPR/Cas system, and is reported to serve as the genetic scissors of Cpf1 only relatively recently (Cell, 2015, 163(3): 759-71). The Cpf1 is a nuclease that is driven by a single-stranded RNA, and requires no tracrRNA and has a characteristic of having a relatively small size compared to the Cas9. Also, it is known that the Cpf1 uses a protospacer-adjacent motif (PAM) sequence rich in thymine, and cleaves a double chain of DNA to form a cohesive end. The Cpf1 may be derived from Candidatus Paceibacter, Candidatus Methanoplasma, and the genus Lachnospira, Butyrivibrio, Peregrinibacteria, Acidominococcus, Porphyromonas, Prevotella, Francisella or Eubacterium. However, the present invention is not limited to the above-described examples as long as the Cpf1 has the above-described RNA-guided nuclease activity. In the present invention, the Cpf1 protein may be a recombinant protein.

For example, when the term “recombination” is used to refer to a cell, a nucleic acid, a protein, a vector, or the like, the term refers to the introduction of a heterologous nucleic acid or protein or alteration of a native nucleic acid or protein, or a cell, a nucleic acid, a protein, or a vector, which is modified by cells derived from modified cells. Therefore, a recombinant Cas9 or a recombinant Cpf1 protein may, for example, be made by reconstructing a sequence encoding the Cas9 or Cpf1 protein using a human codon table.

The Cas9 or Cpf1 protein may be in a form in which the protein functions in the nucleus, and may be in a form in which the protein is easily introduced into cells. For example, the Cas9 or Cpf1 protein may be linked with a cell-penetrating peptide or a protein transduction domain. The protein transduction domain may be a TAT protein derived from poly-arginine or HIV, but the present invention is not limited thereto. Because various types of the cell-penetrating peptide or the protein transduction domain are known in the related art in addition to the above-described example of the protein, those skilled in the art may apply various examples to the present invention without being limited to the above example.

Also, a nucleic acid encoding the Cas9 or Cpf1 protein may further include a nuclear localization signal (NLS) sequence. Therefore, an expression cassette which includes the nucleic acid encoding the Cas9 or Cpf1 protein may include an NLS sequence in addition to regulatory sequences such as a promoter sequence for expressing the Cas9 or Cpf1 protein, and the like, but the present invention is not limited thereto.

The Cas9 or Cpf1 protein of the present invention may be linked with a tag favorable for separation and/or purification. For example, a small-peptide tag such as a His tag, a Flag tag, a S tag, a glutathione S-transferase (GST) tag, a maltose binding protein (MBP) tag, or the like may be linked according to purpose, but the present invention is not limited thereto.

In one specific embodiment of the present invention, the step (a) may be performed, which includes (i) preparing a cell line in which a sequence encoding the RNA-guided nuclease is inserted (knockin); (ii) manufacturing a vector, which includes an oligonucleotide including a base sequence encoding the guide RNA and a target base sequence targeted by the guide RNA; (iii) transducing the vector into the cell line to prepare transduced cells; and (iv) culturing the transduced cells.

According to the method for measuring time in cells according to the present invention, the probability (λ) of decreasing the copy number of the intact target sequence per unit cell in the formation of the indels in the target sequence is determined by the composition of the target sequence, a concentration of the RNA-guided nuclease, a concentration of the guide RNA. As a result, a cell line in which the sequence encoding the RNA-guided nuclease is inserted may be prepared to maintain a constant concentration of the RNA-guided nuclease expressed in the cell line.

A degree of the activity of the nuclease may vary depending on the type and/or number of the guide RNA-target sequence pair or the stgRNA sequence present in the introduced cells. The RNA-guided nuclease may be introduced into the cells by means of a plasmid vector or a viral vector, or an RNA-guided nuclease protein itself may be introduced into the cells. In this case, a method of introducing the RNA-guided nuclease is not particularly limited as long as the RNA-guided nuclease may exhibit activity in the cells. For example, the RNA-guided nuclease (e.g., a Cas protein, a Cpf1 protein) may be introduced in a state in which the RNA-guided nuclease is linked with a protein transduction domain, but the present invention is not limited thereto. Various types of protein transduction domains known in the art may be used as the protein transduction domain. As described above, the protein transduction domain may include a TAT protein derived from poly-arginine or HIV, but the present invention is not particularly limited thereto.

The type of cells may be properly selected by those skilled in the art, depending on the type of vector and/or the type of desired cells. Specifically, the cells may be selected from bacterial cells such as Escherichia coli, Streptomyces sp., Salmonella typhimurium, and the like; yeast cells; fungal cells such as Pichia pastoris, and the like; insect cells such as Drosophila sp., Spodoptera Sf9 cells, and the like; animal cells such as Chinese hamster ovary cells (CHO), SP2/0 (mouse myeloma), human lymphoblastoid, COS, NSO (mouse myeloma), 293T, bow melanoma cells, HT-1080, baby hamster kidney (131-1K) cells, human embryonic kidney (HEK) cells, PERC.6 (human retinal cells); or plant cells, but the present invention is not limited thereto.

In one embodiment of the present invention, a cell line in which Cas9 is inserted may be prepared by inserting a SpCas9 sequence in a transcriptionally active region of HEK293 cells using an FLP recombinase.

Next, a vector, which includes an oligonucleotide including a base sequence encoding the guide RNA and a target base sequence targeted by the guide RNA, may be constructed. According to one embodiment of the present invention, a vector, which includes a base sequence encoding the guide RNA and a target base sequence targeted by the guide RNA, may be constructed. The guide RNA may be two or more different RNAs. In this case, a vector library including two or more vectors, which include base sequences encoding two or more guide RNAs and target base sequences targeted by the respective guide RNAs, may be constructed.

In the present invention, the term “library” refers to a pool (or a population) which includes two or more substances of the same kind having different characteristics. Therefore, an oligonucleotide library may be a pool including two or more oligonucleotides having different base sequences, for example, two oligonucleotides having different guide RNAs, PAM sequences, and/or target sequences, and a vector library (e.g., a viral vector library) may be a pool including two or more vectors having different sequences or constituents. For example, the vector library may be a pool of vectors for each oligonucleotide of the oligonucleotide library, that is, a pool of two or more vectors having different oligonucleotides, which constitute the corresponding vector. A cell library may be a pool of two or more types of cells that have different characteristics, specifically, for the purpose of the present invention, cells that have different oligonucleotides included in respective cell types, for example, a pool of cells having different numbers and/or types (especially, types) of the introduced vectors. In the present invention, because an object of the present invention is to provide a method for measuring the lapse of time in cells using a cell library into which a composition for editing a gene is transduced, there may be two or more types of the oligonucleotides, vectors (e.g., viral vectors) and cells, which constitute each of the libraries. In this case, the upper limits thereof may not be limited as long as the method for measuring time works normally. For example, there are 10,000 types of the oligonucleotides, vectors (e.g., viral vectors) and cells.

In the present invention, the term “oligonucleotide” refers to a substance in which several to hundreds of nucleotides are linked via phosphodiester bonds. For the purpose of the present invention, the oligonucleotide may be double-stranded DNA. The oligonucleotide used in the present invention may have a length of 20 to 300 bp, specifically a length of 50 to 200 bp, and more specifically a length of 100 to 180 bp. In the present invention, the oligonucleotide may include a guide RNA-encoding base sequence and a target base sequence. The oligonucleotide of the present invention may include a self-targeting guide RNA-encoding sequence. Also, the oligonucleotide may include an additional sequence to which a primer can bind so that the oligonucleotide can be amplified by PCR.

Specifically, the guide RNA in the single oligonucleotide may be cis-acting on the target base sequence present adjacent to the guide RNA. That is, the guide RNA may be designed to determine whether or not the adjacent target base sequence is cleaved.

The oligonucleotide is introduced into cells so that the oligonucleotide can be integrated into a chromosome.

The design of the oligonucleotide may be freely performed by those skilled in the art for the purpose of measuring an indel frequency of a target sequence and predicting time from the indel frequency. For example, the oligonucleotide may form a pair with a sequence having target activity for a certain guide RNA sequence. Also, the oligonucleotide may form a pair with a sequence having an off-target activity for the guide RNA sequence. For example, a sequence completely complementary to the guide RNA sequence, specifically, a crRNA sequence, or a sequence complementary to some of the guide RNA sequence, which has some mismatched bases, may be designed. Also, a stgRNA sequence having the same properties as both of the guide RNA and the target sequence may be designed.

Also, a person having skill in the art may allow an additional constituent to be included in the oligonucleotide in order to perform indel analysis to measure time according to the present invention. For example, the oligonucleotide may include one or more selected from the group consisting of a direct repeat sequence, a poly-T sequence, a barcode sequence, a constant region sequence, a promoter sequence, and a scaffold sequence, but the present invention is not limited thereto.

The oligonucleotide may consist of a base sequence having a length as described above, specifically a length of 100 to 200 bases, but the present invention is not limited thereto. In this case, the length of the oligonucleotide may be adjusted by those skilled in the art, depending on the type of RNA-guided nuclease used, the purpose of analysis, and the like.

Meanwhile, the above-described oligonucleotide may include a target sequence and a guide RNA-encoding sequence in sequential order in a 5′ to 3′ direction. On the contrary, the oligonucleotide may be designed to include a guide RNA-encoding sequence and a target sequence in sequential order in a 5′ to 3′ direction.

For example, the oligonucleotide includes a target sequence and a guide RNA-encoding sequence, and specifically may further include a barcode sequence, a PAM sequence, a poly-T sequence, a direct repeat sequence, a constant region sequence, whose sequential order is not limited.

Also, the oligonucleotide may include a stgRNA-encoding sequence, and specifically may further include a barcode sequence, a PAM sequence, a poly-T sequence, a direct repeat sequence, a constant region sequence, whose sequential order is not limited.

In addition, the oligonucleotide may further include a scaffold sequence that is adjacent to the guide RNA-encoding sequence or the stgRNA-encoding sequence to assist the binding of the RNA-guided nuclease.

Furthermore, the oligonucleotide may further include a promoter sequence at a 5′-terminal region for expression. In embodiments of this specification, a U6 promoter known to maintain constant expression of non-coding RNA for a long period of time was used to maintain constant expression of the guide RNA or stgRNA.

Further, as described above, the oligonucleotide may further include a primer attachment sequence enabling PCR amplification at the 5′ and 3′ termini in addition to the above-described constituents, but the present invention is not particularly limited thereto.

The target sequence of the present invention may have a length of 10 to 100 bp, specifically a length of 20 to 50 bp, and more specifically a length of 23 to 34 bp, but the present invention is not particularly limited thereto.

Also, the guide RNA-encoding sequence may have a length of 10 to 100 bp, specifically a length of 15 to 50 bp, and more specifically a length of 20 to 30 bp, but the present invention is not particularly limited thereto.

In addition, the stgRNA-encoding sequence may have a length of 10 to 200 bp, and specifically a length of 80 to 180 bp, but the present invention is not particularly limited thereto.

Furthermore, the barcode sequence refers to a nucleotide sequence used to identify each oligonucleotide. In this specification, the barcode sequence may include two or more repeating nucleotides (AA, TT, CC, GG), but is not particularly limited as long as it is designed to identify each oligonucleotide. In a plurality of oligonucleotides, the barcode sequence may be designed to have at least two different bases in order to identify each oligonucleotide. The barcode sequence may have a length of 5 to 50 bp, but the present invention is not particularly limited thereto.

Subsequently, a vector library (e.g., a viral vector) may be constructed using the oligonucleotide library.

When the vector is a viral vector, a viral library is introduced into cells, and the vector may be then obtained by producing viruses from the cells, and the cells may be infected with the vector library. In this case, methods known in the related art may be used by those skilled in the art to properly perform this process.

In the present invention, the vector may include an oligonucleotide which includes each of a guide RNA-encoding base sequence and a target base sequence, or a stgRNA-encoding base sequence. The vector may be a viral vector or a plasmid vector. Specifically, a lentiviral vector, a retroviral vector, or the like may be used as the viral vector, but the present invention is not limited thereto. For example, known vectors may be freely used by those skilled in the art as long as they are used to achieve the objects of the present invention.

The vector refers to a medium that can deliver the oligonucleotide into a cell, for example, a genetic construct. Specifically, when the vector is present in cells of an individual subject, the vector may include an insert, that is, an insert to which an essential control element is operably linked so that an oligonucleotide can be expressed.

The vector may be prepared and purified using standard recombinant DNA techniques. The kinds of vector may not be particularly limited as long as the vector can act in target cells (e.g., eukaryotes, prokaryotes, and the like). The vector may include a promoter, an initiation codon, and a termination codon terminator. In addition, the vector may appropriately include DNA encoding a signal peptide, and/or an enhancer sequence, and/or an untranslated region at the 5′ and 3′ sides of a desired gene, and/or a selective marker region, and/or a replicable unit, and the like.

In one specific embodiment of the present invention, 24,000 self-targeting guide RNAs (stgRNAs) were designed for quasi-random extraction, and an oligonucleotide library including the same was established. Each of the oligonucleotides constituting the oligonucleotide library has a length of a total of 138 nt (Libraries 1 and 2) or 150 nt (Library 3) bases, and thus includes different stgRNA. Next, each of the oligonucleotides of the oligonucleotide library was cloned into a lentiviral vector to construct a lentiviral vector library, and the lentiviral vector library was expressed in cells to obtain lentiviruses.

The next step includes constructing a cell library including two or more cells in which the vector of the present invention is transduced into a cell line in which the RNA-guided nuclease is inserted.

Specifically, a method for delivering the vector into cells for preparation of the libraries can be achieved by various methods known in the art. For example, the methods may be performed using various methods known in the art, such as a calcium phosphate-DNA co-precipitation method, a DEAE-dextran-mediated transfection method, a polybrene-mediated transfection method, an electroporation method, a microinjection method, a liposome fusion method, Lipofectamine and protoplast fusion methods, and the like. Also, when a viral vector is used, the target product (i.e., the vector) may be delivered into cells by means of infection using viral particles. In addition, the vector may be introduced into cells by gene bombardment, and the like.

The introduced vector may be present as a vector itself in cells or may be integrated into a chromosome, but the present invention is not particularly limited thereto.

The cell library prepared in this specification refers to a cell pool into which an oligonucleotide including a stgRNA-encoding sequence is introduced. In this case, each of the cells may be a cell into which the vector is introduced, specifically a cell into which the vector is introduced with different types and/or numbers of the viruses. However, the method for measuring time through the analysis of the indel frequency according to the present invention is performed using the entire cell library, and the base sequence encoding the guide RNA and the target sequence are introduced in the form of a pair. Therefore, data analysis is possible in a stgRNA-dependent manner without being significantly affected by efficiency of cell infection, variations in the copy number of oligonucleotides, and the like.

Nuclease activity may be exhibited by the oligonucleotide (or stgRNA) of the guide RNA-target sequence pair introduced into the cell library and the RNA-guided nuclease expressed in the cells. That is, DNA cleavage of the introduced target sequence (or stgRNA) by the RNA-guided nuclease may occur, thereby generating indels.

In the present invention, the term “indel” generally refers to a variation in which some bases are inserted or deleted in a DNA sequence. As described above, when a double helix of DNA is cleaved by the RNA-guided nuclease, the indels may be introduced into the target sequence while the cleaved DNA is repaired by a homologous recombination or non-homologous end-joining (NHEJ) mechanism.

The cells cultured in the previous step may be transplanted into an animal, and then cultured. In this case, it is possible to measure the lapse of time in the living animal.

The method for measuring time which has elapsed from a predetermined time point in cells according to the present invention includes [step (b)] harvesting some of the cultured cells at any time point (t) which has elapsed from a predetermined time point, followed by sequencing of a target sequence from the genomic DNA of the cells.

The step may include acquiring a DNA sequence from the cells exhibiting the activity of the introduced RNA-guided nuclease. Such DNA acquisition may be performed using various DNA isolation methods known in the related art.

Because the indels are expected to occur in the introduced target sequence in each of the cells constituting the cell library, bases of the target sequence may be subjected to sequencing (for example deep sequencing), or RNA-sequencing, thereby obtaining the data thereof.

The method for measuring time which has elapsed from a predetermined time point in cells according to the present invention includes [step (c)] measuring an indel frequency (IF) of the target sequence.

As described above, each of the indels occurs in a manner dependent on each of the guide RNA-target sequence pair and the stgRNA sequence. Therefore, the indel frequency may be evaluated as a degree of RNA-guided nuclease activity by the guide RNA-target sequence pair or the stgRNA.

Because the plurality of guide RNA-target sequence pairs or stgRNA sequences may be distinguished by inserting, into each of the oligonucleotides constituting the oligonucleotide library, a particular sequence which is able to distinguish the oligonucleotides, it is possible to perform data analysis by classifying the data based on the distinguished sequences in the step of data analysis. For example, according to the present invention, each of the oligonucleotides was constructed to include a barcode sequence which is designed to include at least two different nucleotides without including two or more repeating nucleotides (i.e., AA, CC, TT, and GG).

The indel frequency of the target sequence has a relationship with the frequency (F) of the copy number of the intact target sequence in the total copy number of the target sequence as shown in the following equation:

F=1−IF

The method for measuring time which has elapsed from a predetermined time point in cells according to the present invention includes [step (d)] calculating any time point using the following equation:

F=1−IF=e^{-λt}(t≥0)

wherein F represents a frequency of the copy number of an intact target sequence in the total copy number of the target sequence, IF represents an indel frequency of the measured target sequence, and λ is a positive constant that represents a probability of generating indels in the target sequence per unit time.

The method for measuring time according to the present invention is based on the fact that the intact target sequence frequency decreases exponentially over time.

The lambda (λ) is a value that represents a probability of generating indels in the target sequence per unit time, or a probability of decreasing the copy number of the intact target sequence per unit time, and refers to a constant that is determined by the composition of the target sequence, concentrations of the RNA-guided nuclease and the guide RNA.

In the present invention, the method may further include estimating a lambda constant (λ), which includes the following steps prior to the step (b):

(i) harvesting some of the cultured cells at a predetermined time point (e);

(ii) sequencing a target sequence from the genomic DNA of the cells;

(iii) measuring a frequency (F) of the copy number of an intact sequence in the total copy number of the target sequence; and

calculating an indel generation probability (λ) of the target sequence per unit time for the given target sequence using the following equation:

F=e^{-λt*}(t*≥0)

wherein F represents a frequency of the copy number of an intact target sequence in the total copy number of the target sequence, λ represents a positive constant, and t* is a positive constant that represents a predetermined time point.

When the concentration of the RNA-guided nuclease and the concentration of the guide RNA (or stgRNA) are constant, the λ of a given target sequence may be experimentally calculated by measuring a frequency (F) of the copy number of an intact target sequence at a certain time point. After the λ of the given target sequence is determined, time may be calculated in a similar manner as in the radiometric dating by measuring an indel frequency (IF) of the target sequence at an unknown time point.

To maintain a constant λ value of a certain target sequence in the relational expression between the indel frequency and time according to one embodiment of the present invention, a cell library was constructed to maintain a constant concentration of the RNA-guided nuclease and a constant expression concentration of the stgRNA.

Based on the fact that the frequency of the copy number of intact target cells decreases in vivo in an exponential manner in the case of the cell library of the present invention, a more accurate and highly predictable method for measuring time may be provided by representing the correlation between the indel frequency value and time at any time point as an exponential-type equation.

In the step (e) of the present invention, any time point may be calculated using the following equation:

F=1−IF=e^{-λ(t-t}^{₀}⁾(t≥0,t₀≥0)

wherein F represents a frequency of the copy number of an intact target sequence in the total copy number of the target sequence, IF represents an indel frequency of the measured target sequence, λ is a positive constant that represents a probability of generating indels in the target sequence per unit time, and to is the latent time taken to express a transgene transduced into cells.

An indel formation process of the present invention includes transducing a composition for editing target genes into cells, followed by culturing of the cells. In this case, after the composition for editing target genes including a guide RNA and a target sequence is transduced into the cells, a certain time is taken until the transgene is expressed. Because there is such a latent time (t₀), an error may occur in time measurement or prediction. Therefore, the method of the present invention may be used to calculate any time point in consideration of an predetermined latent time.

Still another aspect of the present invention provides a system for measuring time in cells, which includes an intracellular indel generation unit including a composition for editing target genes; an intracellular indel frequency measurement unit for sequencing of the target genes; and a time prediction unit for calculating the lapse of time at any time point from a predetermined time point using the measured indel frequency.

Meanwhile, as described above, it is apparent that the definitions and aspects of the terms disclosed above are also applied to the following.

In the system for measuring time in cells according to the present invention, the composition for editing target genes may include a guide RNA, a target base sequence targeted by the guide RNA, and an RNA-guided nuclease.

According to one embodiment of the present invention, the guide RNA and the target base sequence targeted by the guide RNA may be base sequences encoding a self-targeting guide RNA.

The sequencing of the indel frequency measurement unit of the present invention may be performed by deep sequencing.

The time prediction unit of the present invention may calculate any time point using the following equation:

F=1−IF=e^{-λt}(t≥0)

wherein F represents a frequency of the copy number of an intact target sequence in the total copy number of the target sequence, IF represents an indel frequency of the measured target sequence, and λ is a positive constant that represents a probability of generating indels in the target sequence per unit time.

The time prediction unit of the present invention may calculate any time point using the following equation:

F=1−IF=e^{-λ(t-t}^{₀}⁾(t≥0,t₀≥0)

wherein F represents a frequency of the copy number of an intact target sequence in the total copy number of the target sequence, IF represents an indel frequency of the measured target sequence, λ is a positive constant that represents a probability of generating indels in the target sequence per unit time, and to is the latent time taken to express a transgene transduced into cells.

### Advantageous Effects

The method of the present invention is a new synthetic biological clock that enables accurate in vivo measurement of the time which has elapsed from a defined time point to any time point. Through the system of the present invention, time information ranging from hours up to weeks can be accurately recorded in vitro or in vivo in DNA of animal cells and living animals, and the time which has elapsed from a recorded time point can be measured at an unknown time point through DNA sequencing.

Also, when the synthetic DNA clock of the present invention is used, it is possible to accurately record and measure the exposure time of cultured cells to chemicals and the lifespan of living animals remaining after time starts to be recorded in the living animals.

In addition, temporal information regarding various intracellular signal transductions can be recorded and decoded in DNA in the cells using the synthetic DNA clock of the present invention.

### DESCRIPTION OF DRAWINGS

FIG. 1 is a schematic diagram showing a structure of a recombinant vector for preparing Cas9-knockin cells according to the present invention.

FIG. 2A shows the results of determining an expression level of a Cas9 protein while culturing the Cas9-knockin cells for 60 days by Western blot, FIG. 2B is an image of the Cas9-knockin cells observed using a fluorescence microscope (Scale bar=50 μm).

FIG. 3 shows a structure of a self-targeting guide RNA (stgRNA) according to one embodiment of the present invention. A barcode sequence was used to recognize each target sequence.

FIG. 4 is a schematic diagram of an experimental method for predicting time according to the present invention using the Cas9-knockin cells.

FIG. 5 is a schematic diagram showing sampling time points of replicate groups A to H for a lentiviral library.

FIG. 6 shows a distribution of t₀ values predicted from a frequency of an intact target at all analysis time points in the replicate groups A to H.

FIG. 7 shows the data of comparing the suitability of a candidate model for describing changes in frequencies or indels of intact target sequences over time using the replicate groups A to F.

FIGS. 8A to 8C show the data of comparing the suitability of the candidate model for changes in frequencies or indel frequencies of intact target sequences over time. AIC and BIC values calculated using the data (A) for all the replicate groups or the data (C, D) for each of the replicate groups are shown.

FIG. 9 shows dot graphs of intact target sequence frequencies continuously measured for some stgRNAs for 60 days. Each of dotted lines represents an exponential decay curve fitted to the data, and the half-life of the stgRNA-encoding sequence (target sequence) is shown on the graph.

FIG. 10A shows the results of leave-one-out cross-validation (LOOCV), and FIG. 10B shows relative absolute errors for time estimation shown in FIG. 10A. FIG. 10C shows mean relative absolute errors for time estimation calculated from the all-time-point data or the time-point data (>4 days) after 4 days. FIGS. 10D and 10E show nps-weighted mean effects of time values predicted from the mean relative absolute errors calculated from the all-time-point data (FIG. 10D) and the time-point data (FIG. 10E) after 4 days.

FIG. 11 shows the half-lives of the stgRNA-encoding sequences calculated for Libraries 1 and 2.

FIG. 12 shows the correlation of indel frequencies, gamma values, and half-lives between the replicate groups and the libraries: FIG. 12A shows the correlation of indel frequencies between the replicate groups in Library 1, FIG. 12B shows the correlation of indel frequencies between the replicate groups in Library 2, FIG. 12C shows the correlation of gamma values calculated from the different replicate groups in Library 1, FIG. 12D shows the correlation of gamma values calculated from the different replicate groups in Library 2, FIG. 12E shows the correlation of half-lives calculated from the different replicate groups in Library 1, FIG. 12F shows the correlation of half-lives calculated from the different replicate groups in Library 2, FIG. 12G shows the correlation of indel frequencies between the different libraries, and FIG. 12H shows the correlation of half-lives between the different libraries.

FIG. 13 shows the results of comparing nps-weighted means (left) and equal-weighted means (right) of frequencies of intact target sequences between the replicate groups.

FIG. 14A shows mean relative absolute errors for elapsed time prediction at observed time points in each of the replicate groups, and FIG. 14B shows relative absolute errors for time prediction.

FIG. 15 shows the results of analyzing an effect of the number of stgRNA-encoding sequences on the mean relative absolute errors for time prediction based on the intact target frequencies using random sub-sampling (n=10).

FIG. 16A is a schematic diagram showing a structure of a recombinant vector for preparing chemically inducible Cas9-knockin cells (ciCas9-knockin cells) according to the present invention, and FIG. 16B is an image of the ciCas9-knockin cells observed using a fluorescence microscope (Scale bar=50 μm)

FIG. 17A shows a structure of a pair of a sgRNA-coding sequence and a target sequence according to one embodiment of the present invention. A barcode sequence was used to recognize each target sequence. FIG. 17B is a schematic diagram showing an experimental method for predicting time according to the present invention using the ciCas9-knockin cells.

FIG. 18 shows dot graphs of intact target sequence frequencies continuously measured for some target sequences for 60 days in the recording of the elapsed time exposed to a compound in cells. Each of dotted lines represents an exponential decay curve fitted to the data, and the half-life of the stgRNA-encoding sequence (target sequence) is shown on the graph.

FIG. 19 shows a half-life distribution for Libraries 1 to 3.

FIG. 20A shows the results of performing leave-one-out cross-validation (LOOCV) using Library 3, and FIG. 20B shows relative absolute errors for time prediction shown in FIG. 20A.

FIG. 21 shows the results of comparing nps-weighted means of frequencies of intact target sequences in the replicate groups G and H.

FIG. 22 shows mean relative absolute errors when predicting the elapsed time at each time point in each of the replicate groups.

FIG. 23A shows the results of observing Cas9-knockin Library 2 cells seeded in a porous polystyrene scaffold using a microscope (left) and a fluorescence microscope (right) (Scale bar=50 μm). FIG. 23B is a schematic diagram showing an experimental method for predicting the lapse of time in living mice.

FIG. 24 shows the results of measuring intact target sequence frequencies to predict the elapsed time.

FIG. 25 shows the Western blot results (left) showing an expression level of a Cas9 nuclease after the Cas9-knockin cells are infected with Library 2 lentiviruses and is a graph (right) quantifying the expression level of the Cas9 nuclease.

FIG. 26 is a graph showing a concentration of the self-targeting guide RNA in the Library 2 cells.

FIG. 27 is a graph showing the relative absolute errors of time prediction values according to the number of cells analyzed per self-targeting guide RNA.

FIG. 28 is a schematic diagram (left) showing a process of screening for guide RNAs having low genotoxicity and is a graph (right) of comparing half-life distributions of the respective guide RNA sets.

FIG. 29 is a schematic diagram showing a plasmid (Upper) into which a FLEx switch concept is introduced and a plasmid (Lower) using a responsive promoter that is responsive to specific biological events.

FIG. 30 is a schematic diagram showing a FLEx recombination process.

FIG. 31 shows the results of determining the expression of a fluorescent protein according to Wnt signal transduction, inflammatory response, and heat induction as a specific life phenomenon.

FIG. 32 shows the results of determining the expression of the fluorescent protein after injection of LiCl with varying concentrations.

FIG. 33 is a graph showing an indel frequency of the self-targeting guide RNA after a Wnt signal is induced in Wnt-responsive FLEx DNA clock library cells.

### BEST MODE

Hereinafter, the present invention will be described in further detail with reference to exemplary embodiments thereof. However, it will become apparent to those having ordinary skill in the art that these exemplary embodiments are merely intended to illustrate the present invention, and are not to be construed as limiting the scope of the present invention.

### Example 1: Exponential Decay of Intact Target Sequence Induced by CRISPR-Cas9 Nuclease

To record the lapse of time in in vivo cells, a CRISPR-Cas9 system consisting of Cas9 and a single-chain guide RNA (sgRNA) was used to form indels. When the concentrations of the Cas9 and the sgDNA were constantly maintained, it was assumed that an indel generation rate of a target sequence per unit time in individual cells was constant. In this specification, this is indicated by lambda (λ). When one target sequence is introduced into one cell, an indel generation response occurs individually in individual cells, and the generation of respective indels in the target sequence is an independent event.

In this case, the indel generation rate or a decrease rate (λ) of the copy number of an intact target sequence in the entire cell population is linearly proportional to the copy number (Nₜ) of the intact target sequence at time t, and may be indicated by the following equation:

$$\frac{d {N}_{t}}{d t} = - λ {N}_{t} ( λ > 0 ) \qquad [ Equation 1 ]$$

A definite integral equation for time tin Equation 1 is as follows:

[Equation 2]

Fₜ=e^{-λt}

wherein Fₜ=Nₜ/N₀ represents a ratio or relative frequency (hereinafter referred to as “frequency”) of the copy number of an intact target sequence in the total copy number of the target sequence at the time point t, and N₀ represents the initial copy number (time point 0) of the intact target sequence. As shown above in Equation 2, Fₜ complies with the exponential decay used for radiometric dating.

The probability (λ) of decreasing the copy number of the intact target sequence per unit cell is determined by the sequence composition of the target sequence, and the concentrations of Cas9 and guide RNA when the target sequence is introduced by lentiviral transduction. Therefore, when the expression levels of the Cas9 and sgRNA are constantly maintained, λ is determined by the composition of the target sequence.

### Example 2: Stable Maintenance of Concentrations Cas9 and Guide RNA in Cells

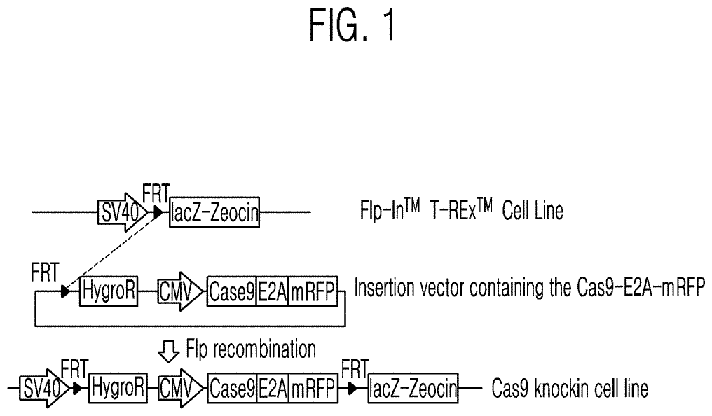

To constantly maintain a concentration of Cas9 in cells, a Streptococcus pyogenes-derived Cas9 (SpCas9)-knockin cell line was used. This cell line was prepared by injecting a CMV promoter-Cas9-E2A-mRFP sequence into transcriptionally active regions of HEK293 cells (Flp-In™ T-REx™ cells) modified using an FLP recombinase (FIG. 1).

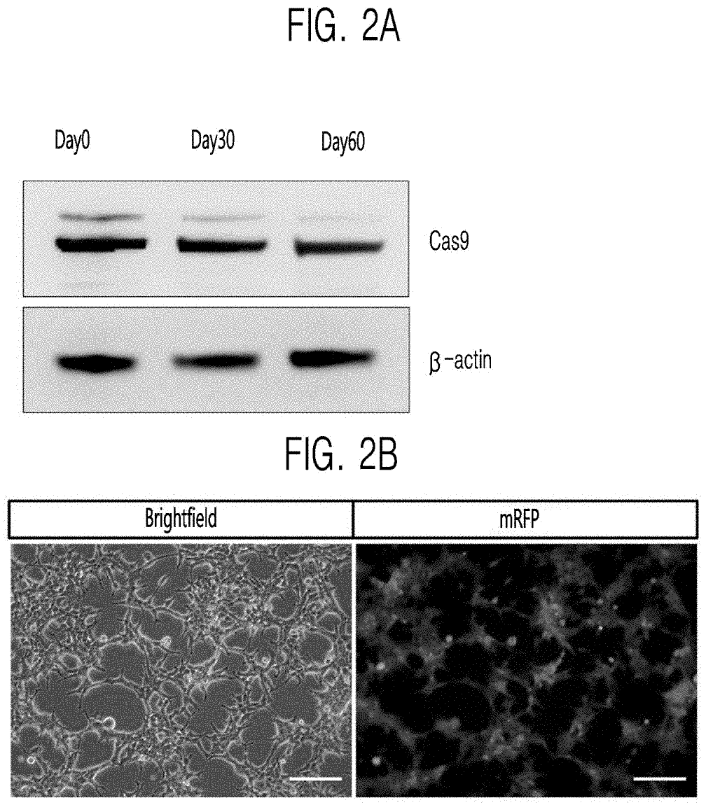

It was confirmed whether the concentration of the Cas9 protein expressed in the Cas9-knockin cells was constantly maintained while culturing the Cas9-knockin cells in the presence of hygromycin for 2 months. From the results of Western blot analysis, it was confirmed that the same amount of Cas9 protein was expressed for 60 days in the cells (FIG. 2A), and confirmed that mRFP translated with Cas9 was uniformly expressed in all the cells in the cell population when observed using a fluorescence microscope (FIG. 2B). From the results, it can be seen that the expression level of SpCas9 was constantly maintained in the Cas9-knockin cells for a long period of time.

Meanwhile, a U6 promoter known to maintain the constant expression of non-coding RNA for a long period of time (several months) was used to maintain the constant expression of the guide RNA.

### Example 3: Generation of Lentiviral Library of stgRNA-Encoding Sequence

When the concentrations of the Cas9 and guide RNA were constant, lambda (λ) of a given target sequence may be determined experimentally by measuring a frequency of the copy number of an intact target sequence at a known time point. When the λ of the given target sequence is determined, the elapsed time may be calculated by measuring an indel frequency (IF) in the target sequence at an unknown time point using a method similar to radiometric dating. The frequency (F) of the intact target sequence is calculated by F=1−IF.

However, when only one guide RNA is used, the accuracy of time prediction and an effectively measurable range of time are limited. Therefore, a plurality of guide RNAs and target sequences corresponding to the guide RNAs were used in the present invention to measure the time for various periods of time with high accuracy.

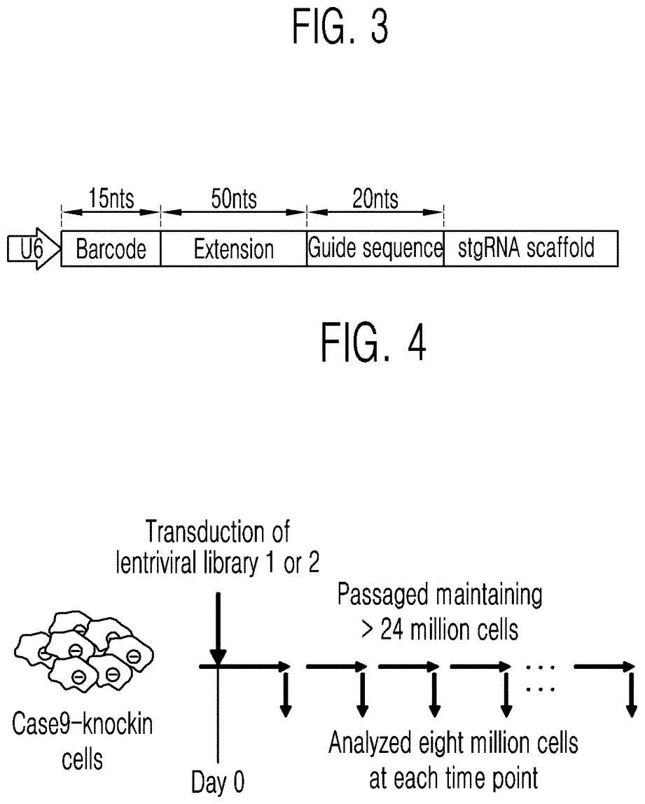

The present inventors have developed a method for determining indel frequencies in several thousands of fused synthetic target sequences using lentiviruses (Korean Patent Laid-Open Publication No. 10-2017-0123581). In such a high-efficiency method for determining indel frequencies, a guide RNA-encoding sequence, a target sequence, and a barcode set for analysis were delivered to 293T cells using a lentiviral vector. Recently, a homing guide RNA or self-targeting guide RNA (stgRNA) system, which may be a guide RNA-encoding sequence and may be a target sequence at the same time, was reported. In the present invention, a self-targeting guide RNA-encoding system and a barcode sequence pair for analysis were used to further simplify the high-efficiency indel frequency analysis system (FIG. 3). Another advantage of using the stgRNA is that it is possible to measure the time for a long period of time because the activity of the stgRNA is reduced compared to common guide RNAs.

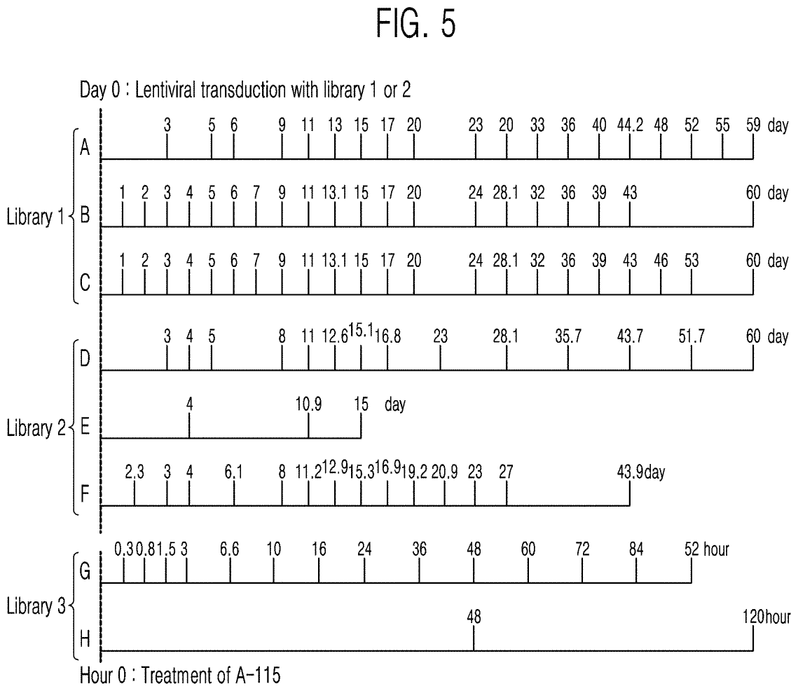

First, Lentiviral library 1 was prepared. Lentiviral library 1 includes 24,000 quasi-randomly selected stgRNA-encoding sequences and barcode sequences corresponding to the stgRNA-encoding sequences. Next, a lentiviral library encoding 24,000 sgtRNAs was transduced into Cas9-knockin cells to prepare a cell library, thereby preparing 3 cell library replicate groups (replicate groups A, B, and C) which were each independently transduced and maintained. These cell libraries were subcultured, and the mean cell number per library was adjusted so that the mean cell number was maintained 1,000-fold higher than the cell number of stgRNA (That is, mean 1,000 cells/stgRNA×24,000 stgRNAs=24 million cells) (FIG. 4). To isolate the genomic DNA, some of the cultured cells were harvested at defined time points (FIG. 5). The target sequence in the genomic DNA was amplified by PCR, and then subjected to deep sequencing in order to evaluate the indel frequency. When the mean indel frequency was measured on day 11 in the replicate group A of Library 1, an indel frequency (IF) of 61% of the stgRNA-encoding sequences was shown to have a low activity of 10% or less.

Therefore, a separate oligonucleotide pool was prepared to construct another lentiviral library (Library 2). Library 2 was designed to include 2,000 stgRNAs having relatively high activity compared to Library 1. Lentiviruses were each independently transduced into Library 2 to prepare 3 replicate groups (replicate groups D, E, and F). The 3 replicate groups were separately subcultured, the mean cell number per library was adjusted so that the mean cell number was maintained 12,000-fold higher than the cell number of stgRNA (that is, 24 million cells) (FIGS. 4 and 5).

### Example 4: Calculation of Latency Period

A binominal distribution B (n, P) may approximate a normal distribution when nP and n(1−P) are sufficiently large. Because the variance of estimator with respect to a true value (parameter) P of the frequency is calculated by P(1−P)/n, the accuracy of estimating the parameter P may be improved when n is a large value and P is an intermediate value (that is, both nP and n(1−P) are large). Based on the frequency p and the total number of implementations n thus observed, it can be seen that the minimum values of np and n(1−p) are able to be used as an indicator for the accuracy of estimating the true value frequency P.

In this example, the number n_{t,i} of the intact target sequences measured for a given certain target sequence I at a given time point t complies with a binominal distribution B(k_{t,i},F_{t,i}), wherein k_{t,i} represents a sequencing read depth of the target sequence i at a given time point t, and F_{t,i} represents a true value of the frequency. Therefore, when both k_{t,i}F_{t,i} and k_{t,i}(1−F_{t,i}) are large, the frequency of the measured intact target sequences may be close to the true value (F_{t,i}). Because the true value (F_{t,i}) is not known in k_{t,i}F_{t,i} and k_{t,i}(1−F_{t,i}), f_{t,i} may be used to estimate F_{t,i}.

In this specification, a smaller value of either k_{t,i}F_{t,i} or k_{t,i}(1−F_{t,i}) is defined as “nps,” and this parameter was used as an indicator for the accuracy of estimating the true value (F_{t,i}) based on the observed value (f_{t,i}).

After the cells were treated with the lentivirus, a predetermined amount of time is required to reversely transcribe a transgene of the lentivirus, insert the transgene into the host genome, and express the transgene of the lentivirus. To estimate the latent time (t₀) as described above, the time was statistically calculated using the data obtained from the replicate groups A to F based on an exponential model. A parameter representing the latent time (t₀) was added to Equation 2, as follows. Then, λ and t₀ were determined using a non-linear least-square method to minimize the residual sum of squares (RSS) weighted with nps with respect to the frequency (F).

Fₜ=e^{-λ(t-t}^{₀}⁾ [Equation 3]

For model fitting, the data with a range of 2%<f_{t,i}<95% was used. In the case of some stgRNAs exhibiting extremely low activity, the frequency F of the intact target sequence did not decrease to 85% or less for 60 days. Therefore, when the intact target sequence frequency measured for certain stgRNAs was greater than or equal to 85% at all measurement time points, the stgRNA-encoding sequence was excluded from the analysis.

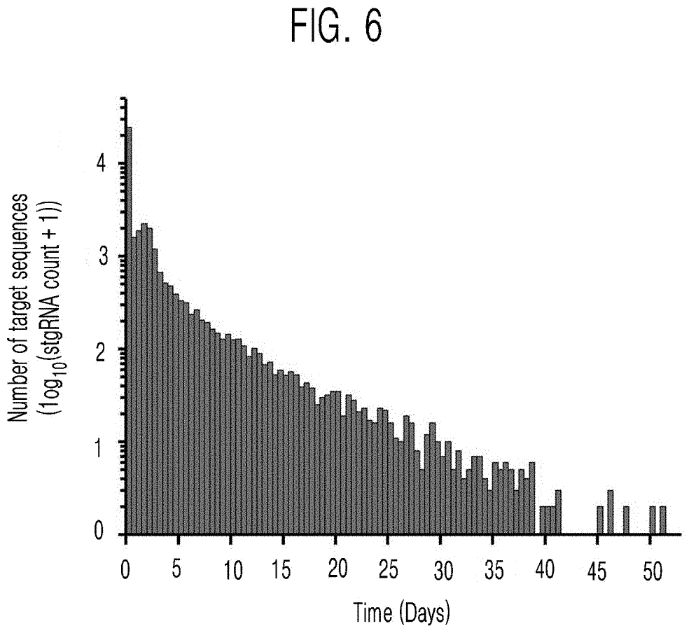

The calculated distribution of a latency period is shown in FIG. 6. In all of the replicate groups A to F, the nps-weighted mean of to truncated by 5% was 1.021 days. This value was used for subsequent analyses.

### Example 5: Verification of Exponential Model

As assumed above, to determine whether the frequency F of the intact target sequence decreased with exponential decay, the exponential model was compared with a linear model, a Gompertz model, and a logistic model. For this purpose, an Akaike information criterion (AIC) and a Bayesian information criterion (BIC) were calculated. To facilitate model fitting, the indel frequency (IF=1−F) of the intact target sequences was used instead of the frequency F of the intact target sequence in the Gompertz model and the logistic model.

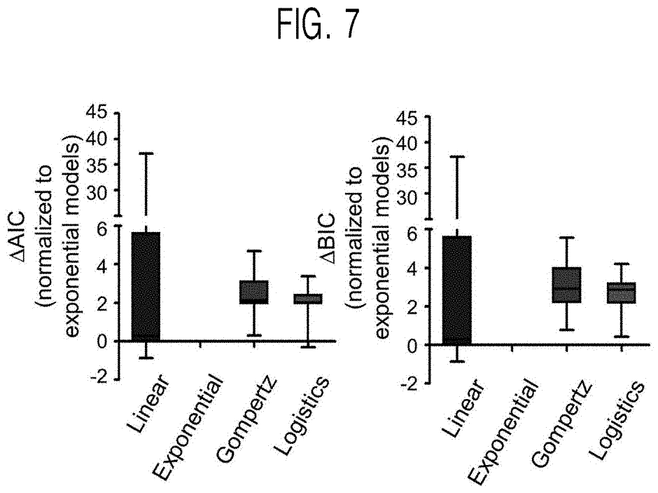

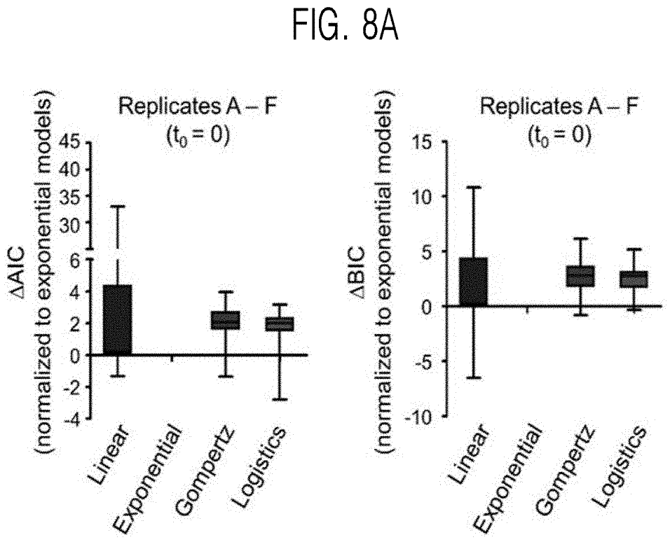

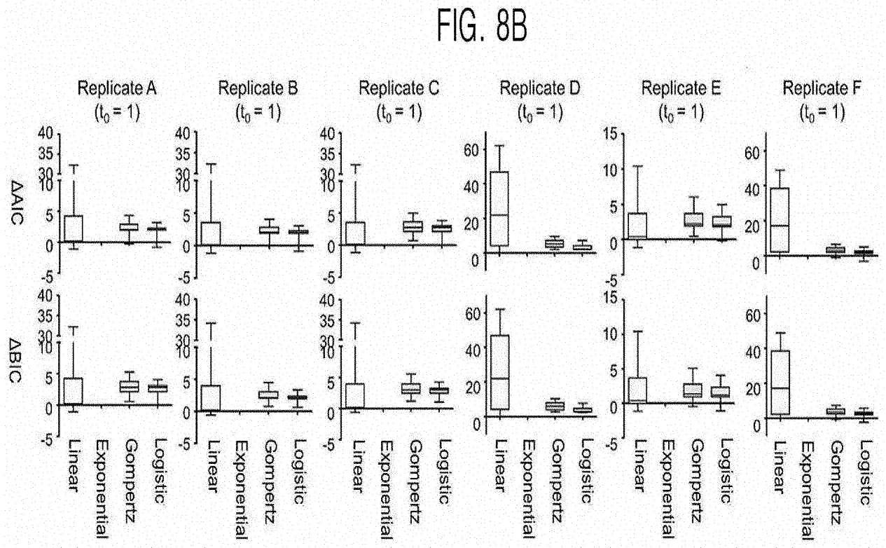

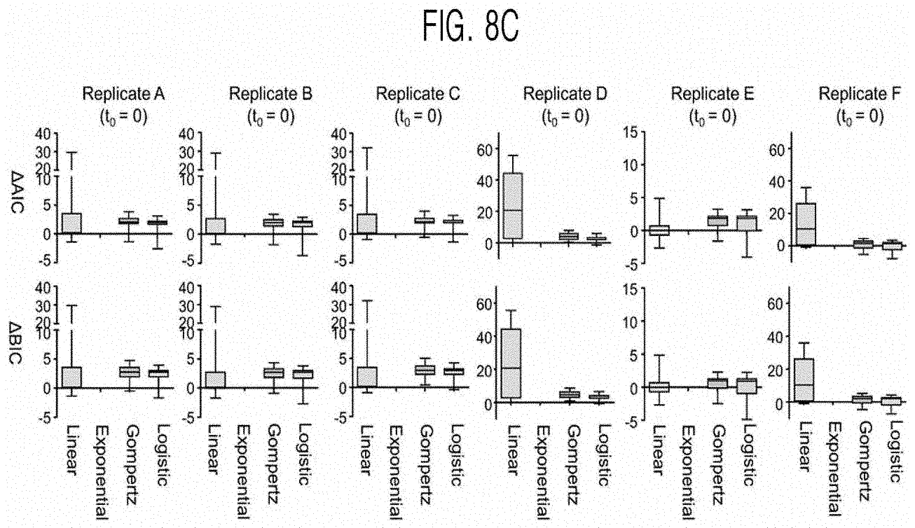

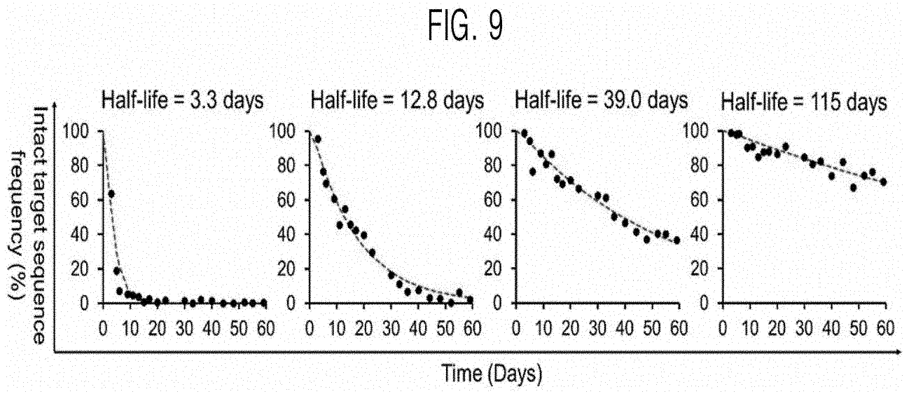

As a result, regardless whether the latency period to was assumed to be either 1.021 days or 0 days, most of the AIC and BIC values in the exponential model were smaller in all the replicate groups A to F, compared to those of the three other models (FIGS. 7, and 8A to 8C). From the results, it can be seen that the intact target sequence frequency decreased according to the exponential decay model. For example, the exponential model fitted to some stgRNAs is shown in FIG. 9.

### Example 6: Measurement of Elapsed Time Using Leave-One-Out Cross-Validation (LOOCV)

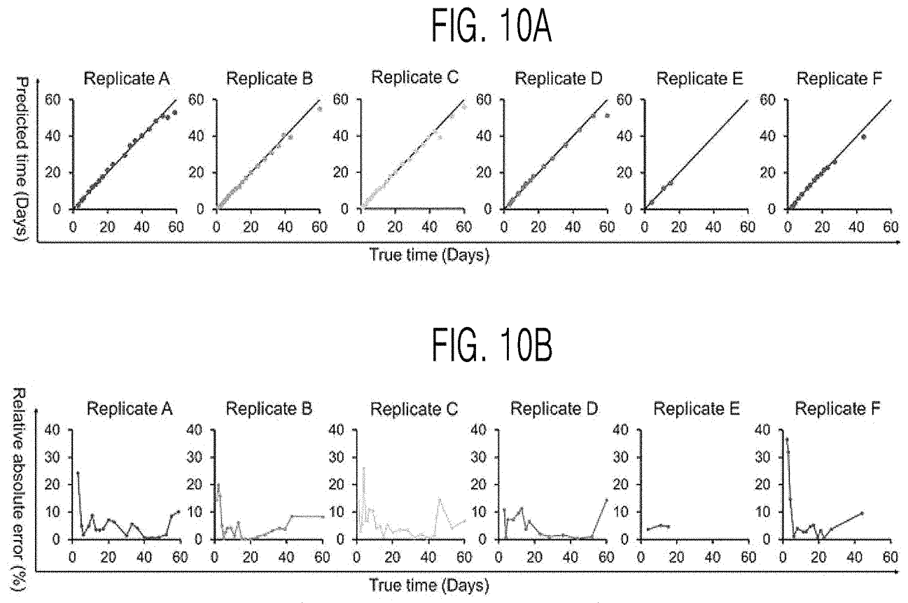

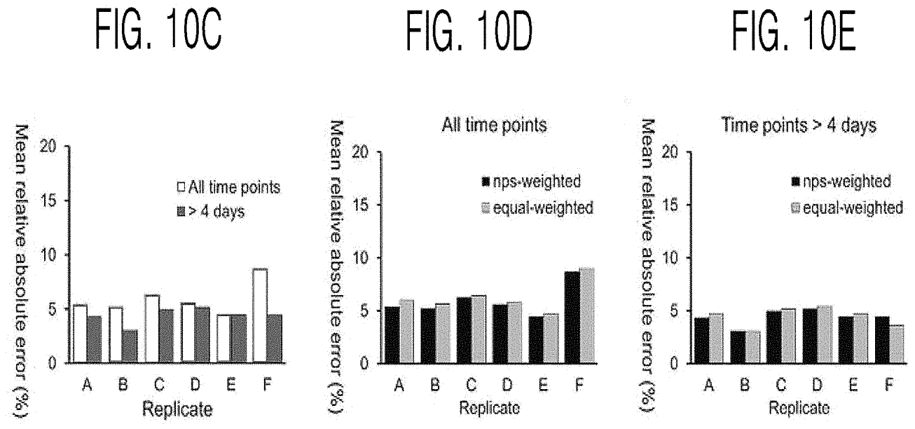

To check whether time was measurable using the exponential decay model of the present invention, leave-one-out cross-validation (LOOCV) was performed. Specifically, one of the measurement time points was selected through experiments, and the elapsed time was predicted using a frequency of an intact sequence at this time point. For this prediction, a non-linear least-square method for minimizing nps-weighted RSS for the frequency was used to calculate λ from a frequency of the intact sequence at another time point. Upon the estimation of the latency period, cases in which the frequency of the intact sequence was an extreme value (f_{t,i}<2% or f_{t,i}≥95%) and when the activity of stgRNA was extremely low (f_{t,i} was greater than or equal to 85% at all of the time points measured for 60 days) were excluded from the analysis. Next, several thousands of estimated time (\hat{t}_{t,i}) values at a certain time point were estimated from several thousands of f_{t,i} values remaining after filtering. To predict the elapsed time (\hat{t}ₜ) at an unknown time point, the quartile (a cut-off value of 25%) nps-weighted mean of several thousands of the \hat{t}_{t,i} values was calculated. From the results, it can be seen that the time prediction for the replicate groups A to F was very accurate (FIG. 10A). In all the replicate groups, the relative absolute errors after 4 days were less than or equal to 20% (FIG. 10B). Also, the time prediction was stabilized after 4 days, and the mean relative absolute errors (MRAEs) after 4 days were in a range of 3.1% to 5.2% in the case of the replicate groups A to F (FIG. 10C). It was confirmed that the MRAEs were in a range of 4.5% to 8.7% at all the time points (mean: 5.9%, and median value: 5.5%). Also, when the accuracy of time prediction based on the calculation of the nps-weighted RSS and nps-weighted mean of the estimated time (\hat{t}_{t,i}) values was compared to the accuracy of time prediction based on the calculation of the equal-weighted RSS and equal-weighted mean of the estimated time (\hat{t}_{t,i}) value, it was confirmed that all the MRAEs at all the time points and the MRAEs after 4 days were lower when an nps-weighted approach was used (FIG. 10D-10E). From the results, it can be seen that the nps-weighted mean method further enhanced the accuracy of time prediction.

### Example 7: Half-Life of Target Sequences

Because half-life (t_{1/2}) was more widely used instead of λ in the case of radiometric dating, Equation 2 may be expressed as follows:

$$F = {e}^{- λ t} = {( \frac{1}{2} )}^{\frac{t}{{t}_{1 / 2}}} , where {t}_{1 / 2} ( Half life ) = \frac{ln 2}{λ} \qquad [ Equation 4 ]$$

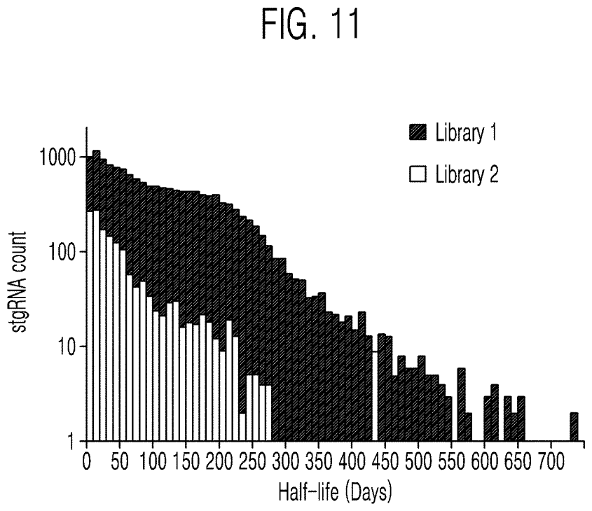

The half-life of each of the stgRNA-encoding sequences was determined from the frequency of the intact sequence at all the measurement time points using the non-linear least-square method as described above. When a certain stgRNA sequence was used for one or more replicate groups, the nps-weighted mean half-life calculated from the half-lives of the replicate groups was selected as an estimated value closest to the real half-life. For Libraries 1 and 2, the half-life associated with the stgRNA ranged from 2.3 to 747 days (median value: 91.5 days, and mean: 113 days) in the case of Library 1 and ranged from 2.7 to 642 days (median value: 34.7 days, mean: 56.6 days) in the case of Library 2 (FIG. 11).

### Example 8: Confirmation of Reproducibility of Recording and Measurement of Elapsed Time Between Different Replicate Groups and Libraries

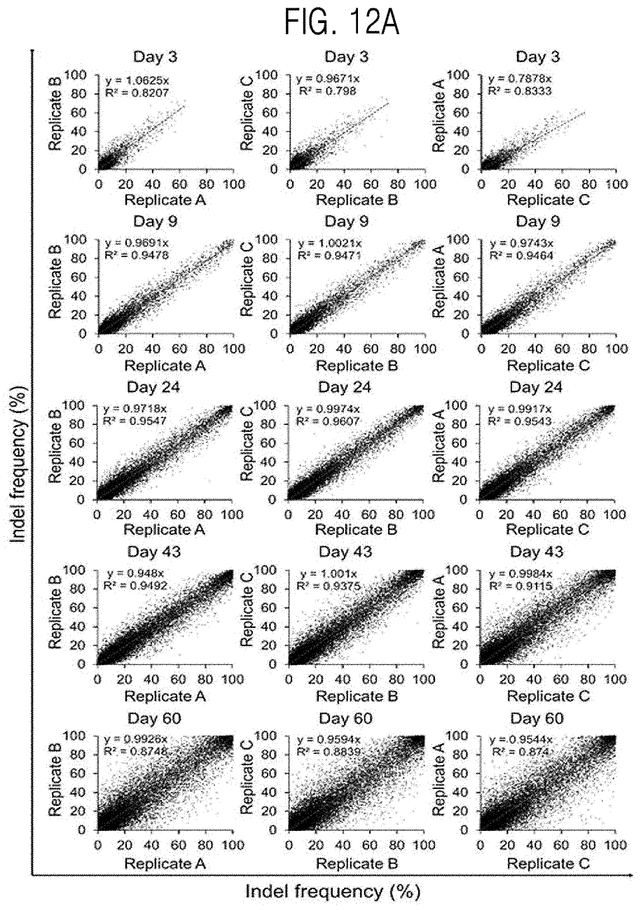

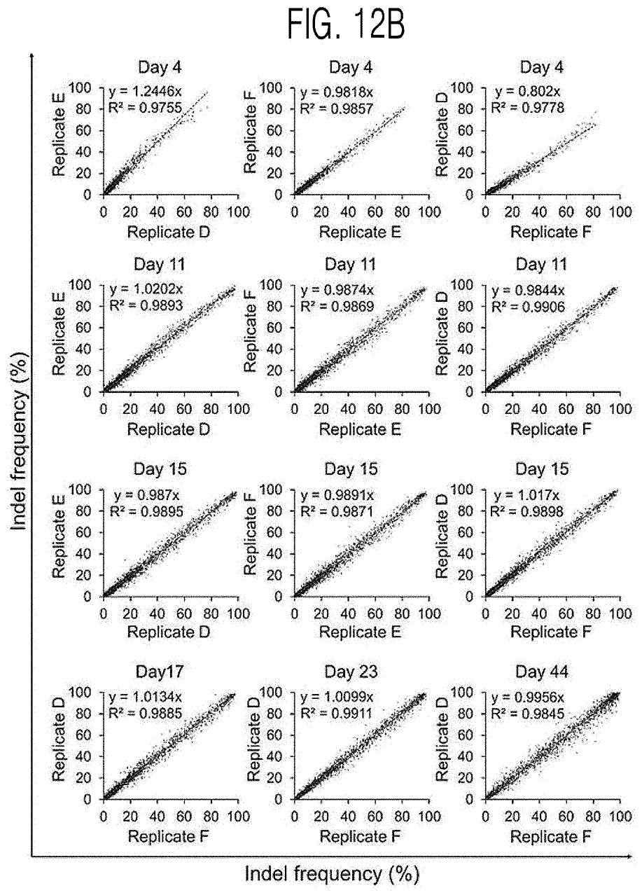

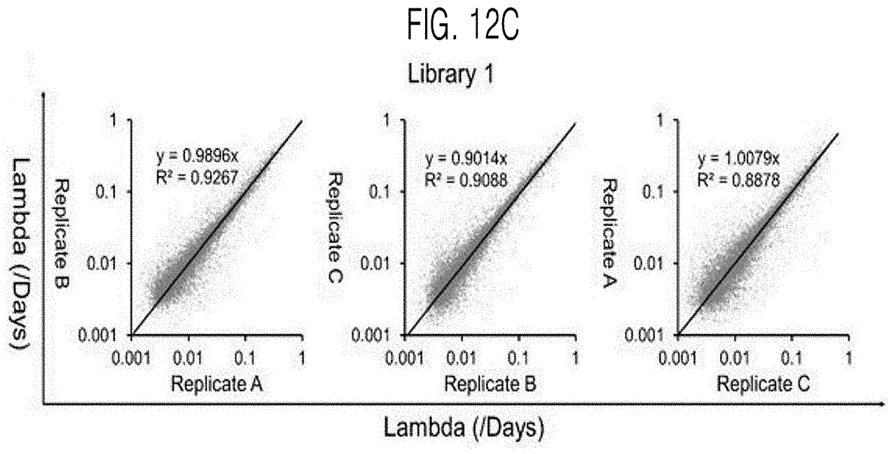

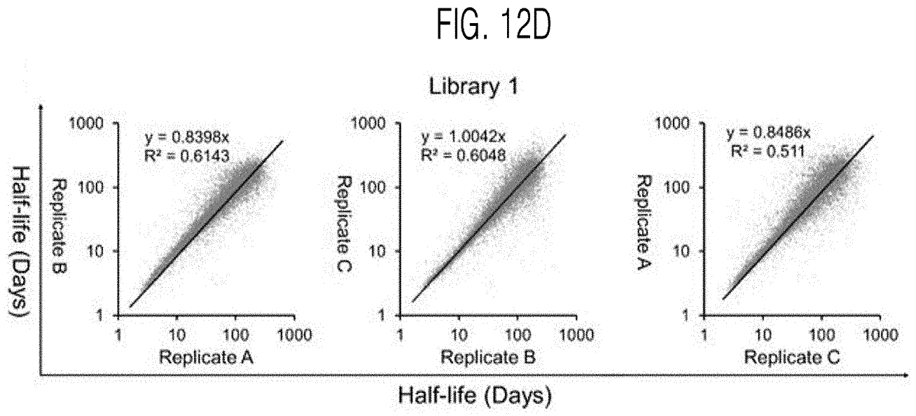

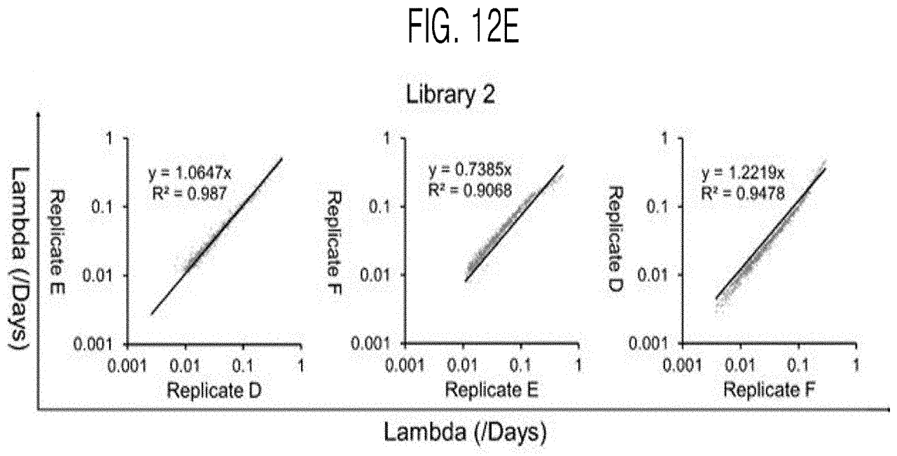

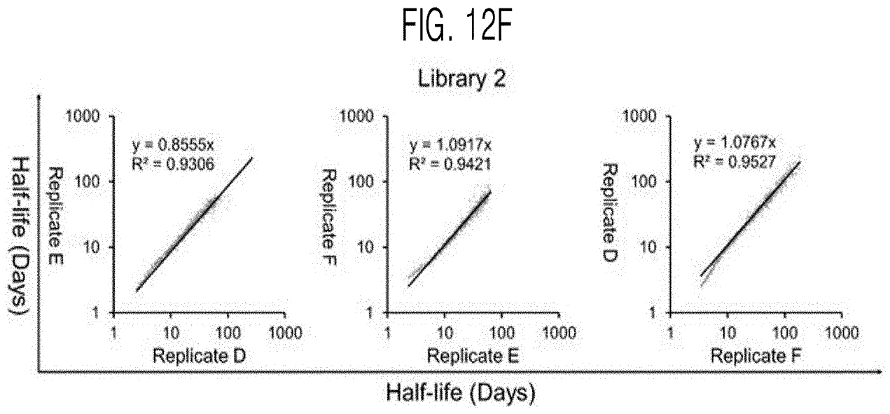

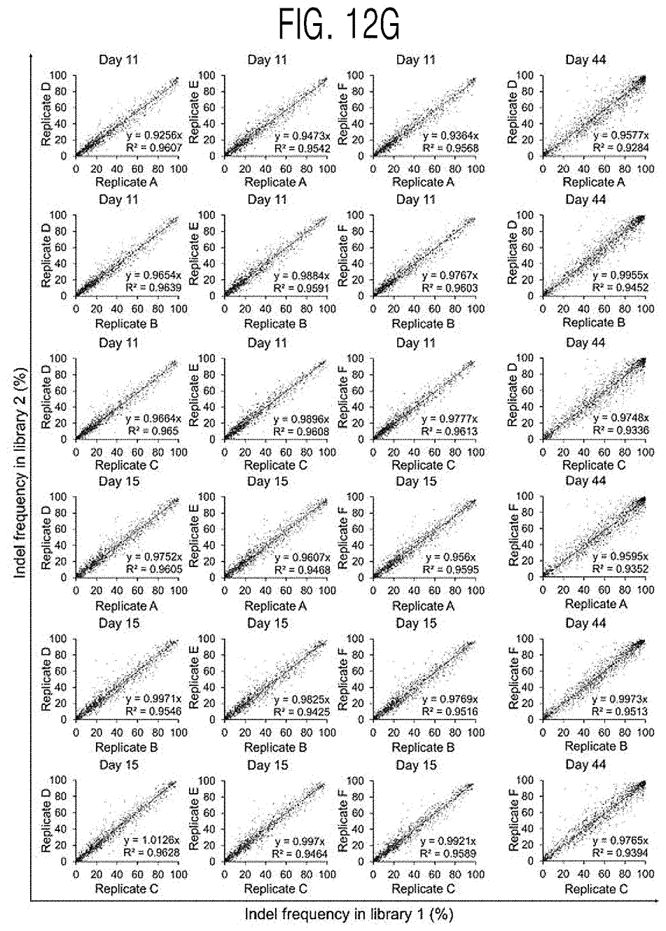

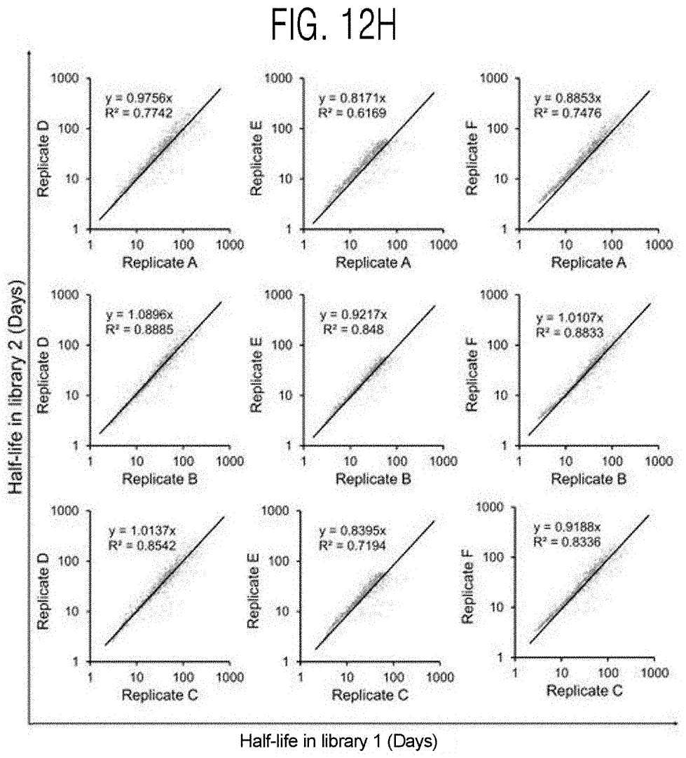

To use the F or IF value as a clock, reproducibility and high correlation between the replicate groups are essential. In Libraries 1 and 2, there was a high correlation in indel frequencies between the replicate groups (FIGS. 12A and 12B). The half-lives and λ values calculated from the intact sequence frequencies (Fs=1−IF) of the different replicate groups were also comparable in the two libraries (FIGS. 12C to 12F).

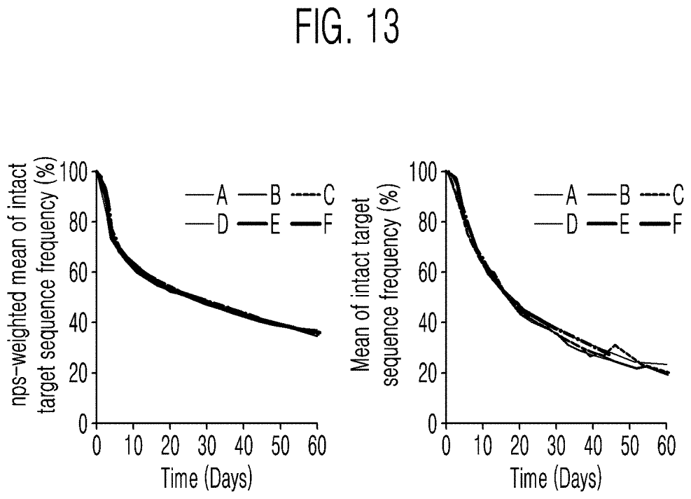

Next, the reproducibility between the different libraries was evaluated. Libraries 1 and 2 shared 1,200 stgRNAs, and the half-lives of 889 of the 1,200 target sequences were determined in all the replicate groups A, B, C, D, E, and F. The indel frequencies were also highly correlated between the replicate groups in the different libraries (FIG. 12G), and the nps-weighted mean and equal-weighted mean of the frequencies (Fs) of the intact target sequences were comparable in all of the 6 replicate groups (FIG. 13). From the results, it can be seen that a decrease rate of the intact sequence frequency for the given stgRNA-encoding sequence was nearly identical and independent in every library batch. Also, although the frequencies (Fs) of the intact sequences were measured only at three time points (4.0, 10.9, and 15.1 days) in the case of the replicate group E, the high correlation between the half-lives and the λ values calculated from the replicate groups of different libraries was also confirmed (FIG. 12H).

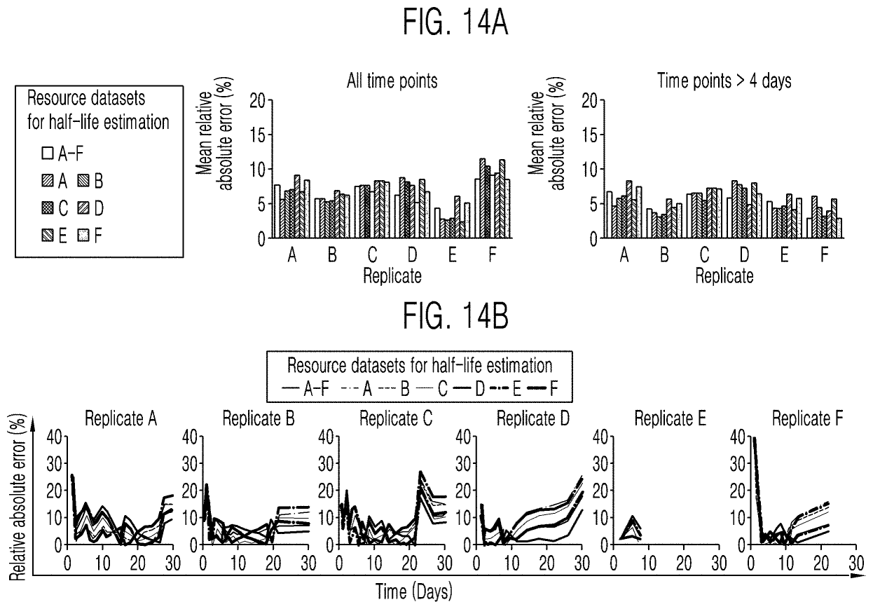

Finally, it was evaluated whether time prediction was reproducible using the 889 shared stgRNAs. When the time which had elapsed from an unknown time point was estimated for any replicate group, the time prediction errors were similar and the mean of the errors decreased by 10% or less after 4 days when the half-lives calculated from another replicate group were used (FIGS. 14A and 14B). From the results, it can be seen that the system of the present invention exhibited high reproducibility and accuracy regardless of the library batches or the replicate groups.

### Example 9: Effect of Decrease in Number of Target Sequences on Accuracy of Time Measurement

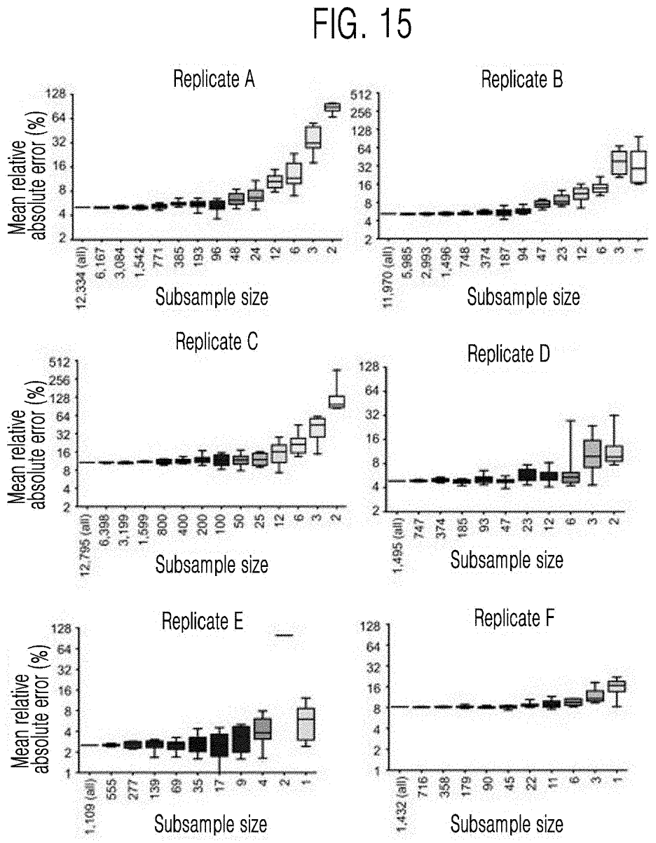

Next, it was checked whether a smaller number of the stgRNAs could be used to measure time. Specifically, the number of stgRNAs was reduced through random extraction from the replicate groups A to F, and MRAE values were calculated for each replicate group. The MRAE values were comparable for almost 100 or 200 stgRNAs. Afterward, the MRAEs drastically changed and increased with a decreasing number of stgRNAs (FIG. 15). From the results, it can be seen that the small-scale libraries including at least 100 or 200 stgRNAs were able to be used for relatively accurate time measurement.

### Example 10: Recording of Elapsed Exposure Time to Chemicals in Cells

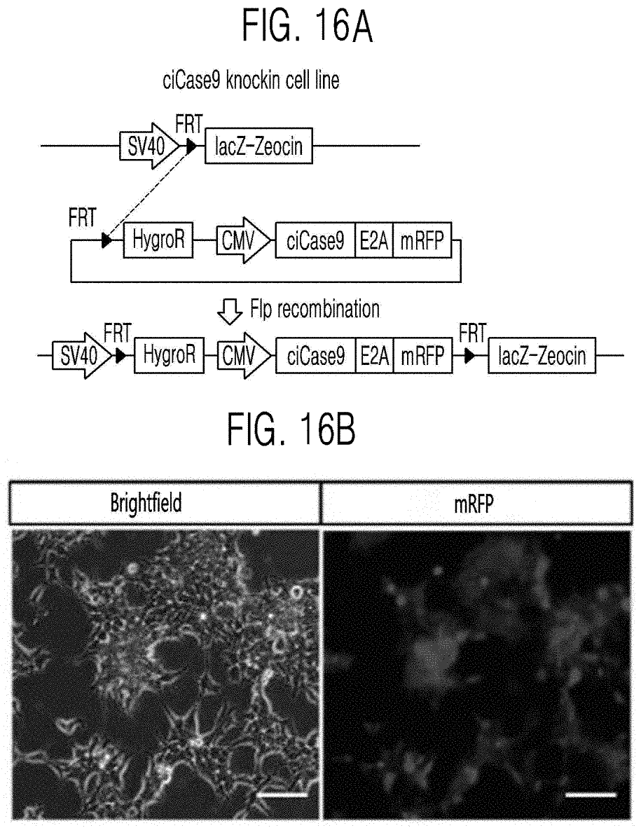

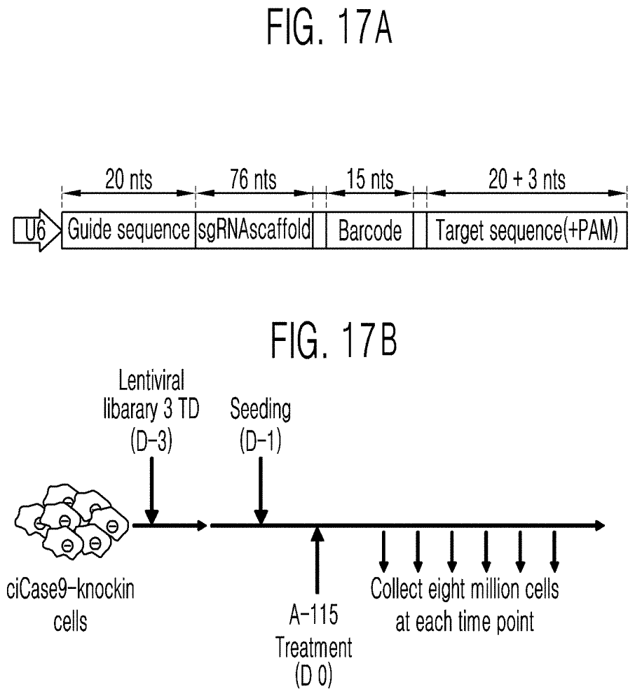

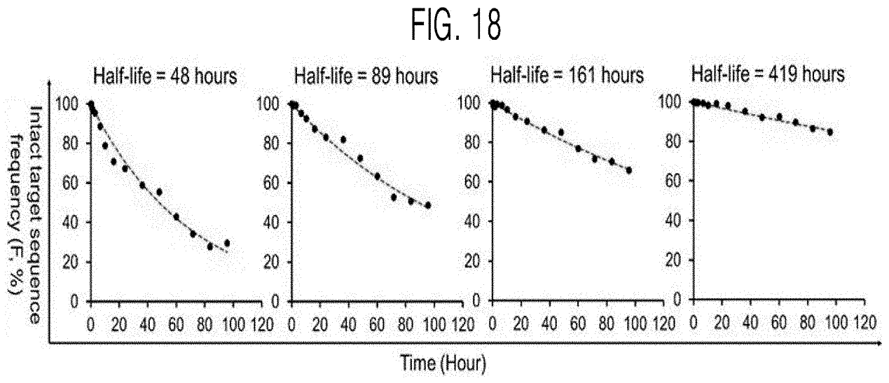

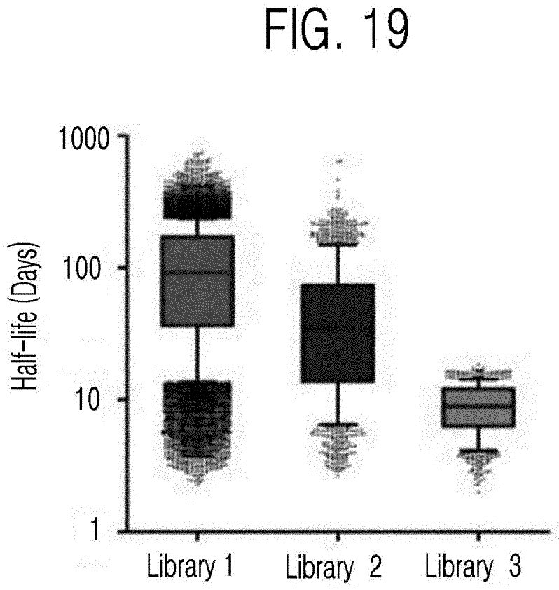

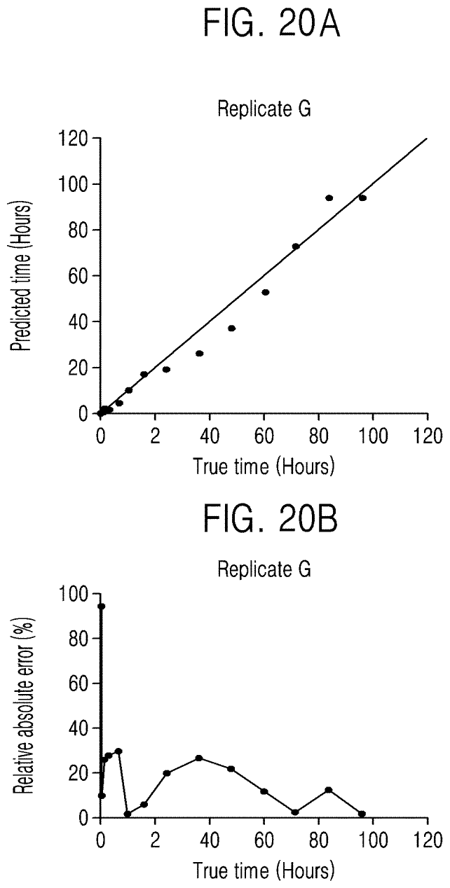

To record the exposure time to chemicals in a DNA sequence, chemically-inducible Cas9 (ciCas9) was used. In this case, the ciCas9 is rapidly activated in the presence of compound A-1155463 (Rose et al., 2017; Rose et al., 2018). First, ciCas9-knockin cells were prepared in the same manner as in the method of preparing the Cas9-knockin cells (FIG. 16). Also, because the stgRNAs exhibited very weak activity compared to the sgRNAs, a library pair including common sgRNA-coding sequences and target sequences corresponding to the sgRNA-coding sequences was separately used instead of the stgRNA-encoding sequences in order to record a relatively short period of time such as several hours (FIG. 17A). Library 3 including a pair of the sgRNA-coding and target sequences was transduced into the ciCas9-knockin cells. The transduced cells were treated with 10 μM A-1155463, and the frequency of the intact sequence was then measured over time (FIGS. 17B and 5). The intact target sequence frequency decreased exponentially over time (FIG. 18). The half-lives calculated in the presence of A-1155463 ranged from 47.9 hours to 442 hours (mean: 219 hours, median value: 214 hours) (FIG. 19). As performed using Libraries 1 and 2, the leave-one-out cross-validation (LOOCV) was performed using Library 3. As a result, the accuracy of time prediction according to the present invention was very high (FIG. 20A), and the relative absolute errors after 50 minutes were less than or equal to 30% (FIG. 20B). From the results, it can be seen that the elapsed exposure time to a chemical could be recorded and measured, and this recording was more accurate after 50 minutes.

When the cells were not treated with A-1155463, the estimated time at day 4 (=96 hours) calculated using the half-lives determined in the presence of A-1155463 was 2.1 hours, which was 46 times shorter than an elapsed time of 96 hours estimated in the presence of A-1155463.

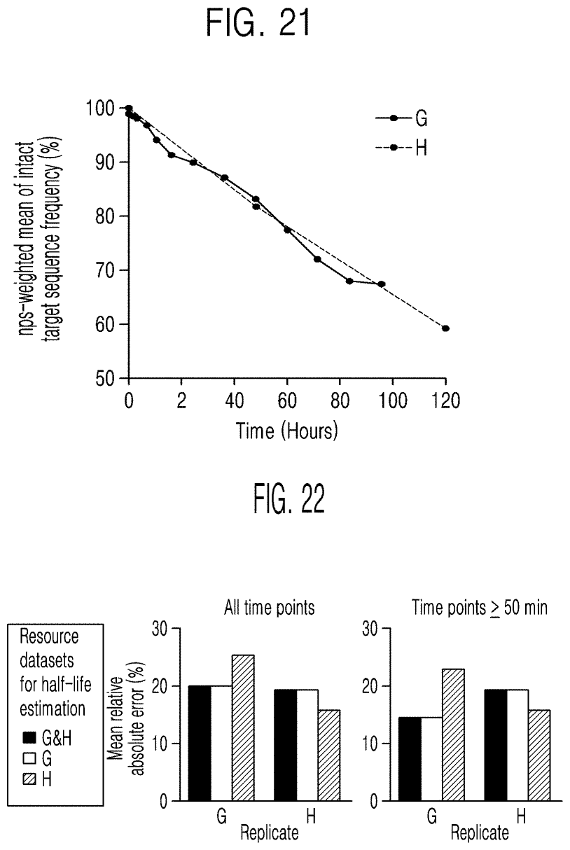

Also, all the nps-weighted means of the intact target sequence frequencies were similar in the replicate groups G and H (FIG. 21). From the results, it can be seen that the ciCas9-induced indel formation rates were comparable between the replicate groups. When the exposure time to A-1155463 at an unknown time point was estimated for any replicate group, the time prediction errors were similar and the mean of the errors decreased by 30% or less when the half-lives calculated from another replicate group were used although only two time points (48 hours, 120 hours) were included in the case of the replicate group H (FIG. 22). From the results, it can be seen that there is high reproducibility between the different replicate groups.

Further, when the time points were calculated at 50 minutes and after that time, the MRAEs were lower than the mean value of all the time points. The results support that the time prediction system of the present invention has high reproducibility and accuracy. As a result, it can be seen that the elapsed exposure time to the A-1155463 compound could be recorded.

### Example 11: Recording of Elapsed Time in Living Mice

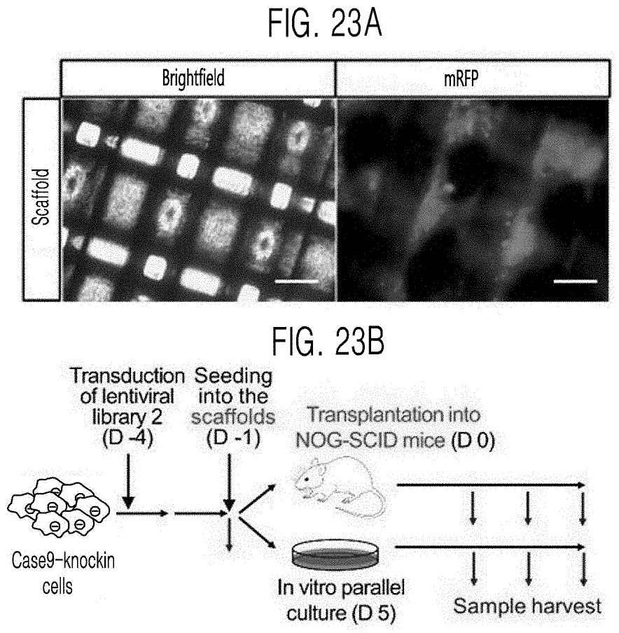

Next, it was checked whether the lapse of time could be recorded in vivo in an animal model. First, the present inventors assumed that, when the Cas9-knockin cells transduced with a library of stgRNA-encoding sequence are delivered into a mouse at a known time point and an intact target sequence frequency is then analyzed, time may be predicted in a live animal after the delivery of the cells. To effectively deliver the cells into a mouse, first, Library 2 was transduced into the Cas9-knockin cells, and the transduced cells were seeded in a porous polystyrene scaffold at a concentration of 1 million cells/scaffold. Two days after the seeding of the cells, it was confirmed, using a fluorescence microscope, that the cells were well attached to the scaffold (FIG. 23A). Then, the scaffold including these cells was subcutaneously transplanted into an NOG-SCID mouse. Thereafter, the mouse was euthanized on days 4, 8, 14, and 21 after the cell transplantation, the scaffold was removed, and then stored at −20° C. until analysis (FIG. 23B). As the in vitro control group under the same conditions, a scaffold including the cells was cultured in vitro.

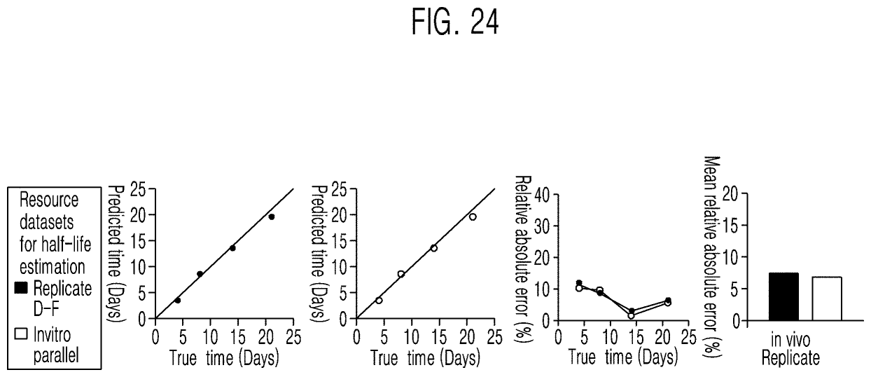

Genomic DNA was isolated from the scaffold, an intact target sequence frequency was evaluated, and the elapsed time was estimated using the half-lives determined through the analysis of the replicate groups D to F in an independent in vitro experiment as described above. As a result, the relative absolute errors of lifespans after the scaffold transplantation, which were measured based on the intact target sequence frequency, were merely 12%, 8.8%, 3.1%, and 6.4% (mean error for a total of 4 time points: 7.6%) on days 4, 8, 14, and 21, respectively (Replicates D to F in FIG. 24). From the results, it can be seen that the elapsed time could be recorded in the mouse by an accurate method.

Also, the errors were comparable when the half-lives determined in a control experiment were used (in vitro parallel in FIG. 24). From the results, it can be seen that the reproducibility of the time recording system of the present invention was independent in each replicate group, and was hardly affected by a subtle environmental difference between the in vitro and in vivo conditions. This indicates that, if the Cas9-induced indel generation was stopped when an animal dies, the lifespan of the animal may be recorded when a time-recording cell is transplanted into the animal at a defined time point.

### Example 12: Confirmation of Maintenance of Concentrations of RNA-Guided Nuclease and Self-Targeting Guide RNA in Cells

When it was assumed that the probability (λ) of decreasing the copy number of the intact target sequence per unit cell was determined by the sequence of the target sequence and the concentrations of the RNA-guided nuclease and guide RNA, it was checked whether the concentrations of the expressed RNA-guide nuclease and self-targeting guide RNA were constantly maintained.

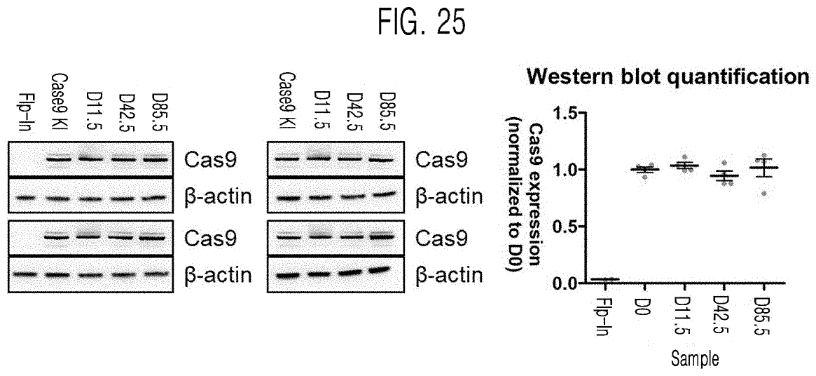

First, to check a concentration of the expressed RNA-guided nuclease, an expression level of the RNA-guided nuclease was checked through Western blotting while culturing the Cas9-knockin Library 2 cells for 85.5 days (FIG. 25). In the Western blot, Flp-In™ T-REx™ cells were used as Flp-In, and Cas9-knockin cells was used as Cas9 KI. Each of D11.5, D42.5, and D85.5 represents the number of days after the Cas9-knockin cells were infected with a Library 2 lentivirus (Flp-In sample: n=2, and other samples: n=4). As shown in the quantification graph, it was confirmed that the Cas9 nuclease expression level was constantly maintained.

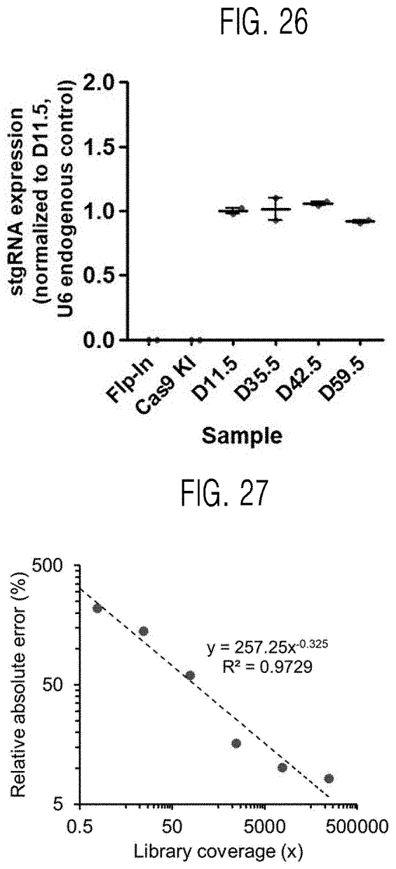

To check a concentration of the self-targeting guide RNA in a cell library, small RNA was extracted from each of Flp-In cells, Cas9-knockin cells, Library 2 cells (samples at days 11.5, 35.5, 42.5, and 59.5) (miRNeasy Mini Kit, QIAGEN) to synthesize cDNA. Thereafter, the cDNA was quantified using qPCR (SYBR Green PCR Master Mix, ThermoFisher) (FIG. 26). Small RNA was extracted from each of the samples to measure a concentration of the self-targeting RNA by RT-qPCR (n=2 per sample). U6 snRNA was used as the endogenous control group. Primers used in qPCR are listed in the following Table 1. As shown in the quantification graph, it was confirmed that the concentration of the self-targeting guide RNA was constant in Library 2 cells for 59.5 days.

TABLE 1

| | | | SEQ ID |
|---|---|---|---|
| Usage | Name | Sequence | NO: |
| stgRNA | stgRNA_scaffold_pF1 | GGGTTAGAGCTAGAAATAGCAAGTT | 21 |
| expression | | AACC | |
| quantification | stgRNA_scaffold_pR1 | CCGACTCGGTGCCACTTTTTC | 22 |
| | U6_endogenous_ctrl_pF1 | CTCGCTTCGGCAGCACA | 23 |
| | U6_endogenous_ctrl_pR1 | AACGCTTCACGAATTTGCGT | 24 |

### Example 13: Confirmation of Relationship Between the Number of Observed Cells and Accuracy of Time Prediction

To measure time using a cell library, a sufficient number of the cells have to be analyzed for different types of self-targeting guide RNAs. In this case, the reliability of indel frequency data may be ensured. Also, the accuracy of time prediction to be analyzed may be enhanced using the indel frequency data having high reliability. Therefore, to check the minimum number of cells required to achieve a time prediction accuracy higher than a certain level, a time prediction error value was measured according to the number of the observed cells.

After the Cas9-knockin cells were infected with the Library 2 lentivirus, samples were analyzed 14.5 days later. A time prediction error value was measured using the half-life information on a final list of half-lives (FIG. 27). The library coverage (x) refers to the number of cells analyzed per self-targeting guide RNA. When 0.12 cells per self-targeting guide RNA, which was the minimum number of cells, were analyzed, the relative absolute error value was very high at 552.0%. When 120,000 cells per self-targeting guide RNA, which was the maximum number of cells, were analyzed, the relative absolute error value was 8.2%, indicating excellent time prediction accuracy. Meanwhile, when 1,200 cells per self-targeting guide RNA were analyzed, the relative absolute error value was 16%. Therefore, it was confirmed that a significantly accurate time prediction value could be derived.

### Example 14: Screening of Self-Targeting Guide RNAs with Low Cytotoxicity

To measure time using a cell library, it is essential to form indels by double strand breaks performed at a constant rate in cells. However, continuous double strand breaks have a risk of causing an off-target effect by which other genome sequences similar to the sequence of each of the self-targeting guide RNAs may be damaged. Screening of the self-targeting guide RNAs used in this experiment was performed to minimize such genotoxicity and maximize stability.

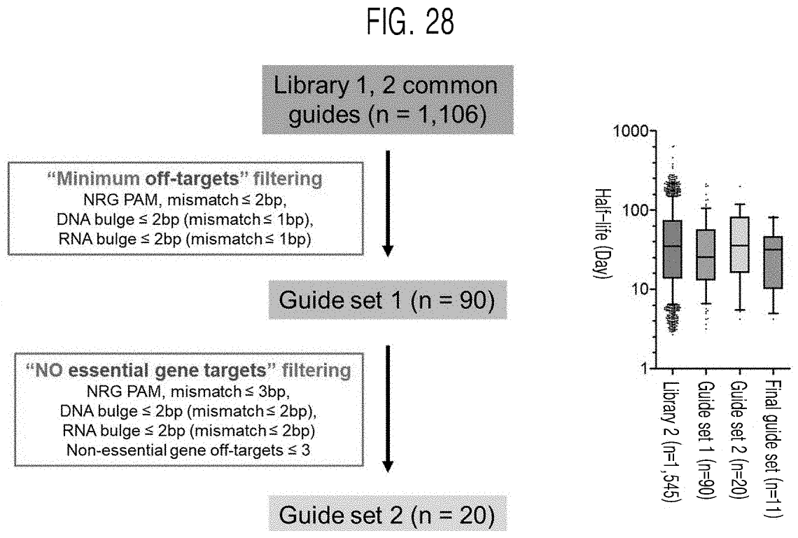

First, filtering was performed to remove self-targeting guide RNAs, which had a sequence similar to the sequence present on the human genome, from the common guide RNAs in Libraries 1 and 2 using the code of a Cas9-OFFinder (Bae S et al., Bioinformatics (2014)) web tool that finds potential off-target sites of Cas9 based on the base sequences. The number of genomic off-target sites, which were completely identical to the human genome sequence or had 1- and 2-bp mismatches based on the NRG PAM sequence when the 20-nt guide base sequence in the components of the library base sequence was compared with the human genome sequence, was analyzed under the filtering conditions used. Also, the number of the genomic off-target sites, which had a completely identical sequence or had a 1-bp mismatch even under the DNA bulge and RNA bulge 1- and 2-bp mismatched conditions, was deduced. Only 90 self-targeting guide RNAs (Guide set 1) in a decreasing order of the total number of the off-target sites under the first filtering condition, were used in the next filtering process (FIG. 28).

Next, the guide RNAs having a sequence similar to the sequences of genes essential for survival (Hart T et al., EMBO Molecular Systems Biology (2014), Hart T et al., Cell (2015)) were removed from the 90 guide RNAs remaining after the first filtering. The number of genomic off-target sites, which were completely identical to the base sequences of the genes essential for survival or had 1-, 2-, and 3-bp mismatches, or had a completely identical sequence or had 1- and 2-bp mismatches even under the DNA bulge and RNA bulge 1- and 2-bp mismatched conditions based on the NRG PAM sequence when the library 20-nt guide base sequence was compared with the base sequences of the genes essential for survival under the filtering conditions used, was analyzed. Twenty self-targeting guide RNAs (Guide set 2) in a decreasing order of the total number of the off-target sites under the second filtering condition were selected, and the final 20 self-targeting guide RNAs had 3 or less off-target sites rather than the genes essential for survival.

When the distributions of half-lives of the self-targeting guide RNAs belonging to Library 2 and Guide sets 1 and 2 were compared, there was no great difference in the distributions of half-lives. Therefore, it was confirmed that the RNAs having high stability were finally screened.

### Example 15: Construction of FLEx DNA Clock Library System for Generalization of DNA Clock

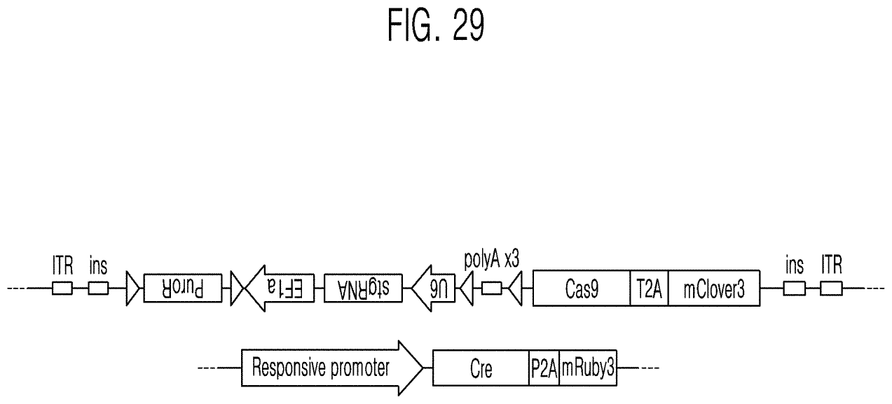

To extend the applicability of the cell library as a DNA clock, a generalized system capable of measuring a variety of other biological phenomena without using the Cas9-knockin cells or the ciCas9-knockin cells was constructed. A FLEx switch concept using Cre-mediated recombination was introduced to allow the system to induce the expression of the Cas9 nuclease in response to specific biological events (FIG. 29) (Schnutgen F et al., Nature Biotech (2003), Andersson-Rolf A et al., Nature Biotech (2017)).

As a vector using the FLEx DNA clock library system, a vector was newly designed based on the Sleeping Beauty (SB) transposon. This was because a polyA sequence cannot be used in the libraries using lentiviruses, such as conventional Libraries 1, 2, and 3, and it is inappropriate for encoding a cassette having a size of 8 kb or more. Therefore, because ITR base sequences required for SB transposition are present at both ends of the cassette, the cassette may be allowed to be inserted into a genomic base sequence in cells by an SB transposase. An ins (insulator) sequence positioned inside the ITR base sequences was added to adjust an expression level of the Cas9 nuclease to a similar level in each cell after the DNA clock is activated (Loveless T B et al., BioRxiv (2019), Liu M et al., Nature Biotech (2015)). In FIG. 29, each of a triangle between ins and PuroR and a triangle between U6 and polyA represents a lox2272 sequence, each of a triangle between PuroR and EF1a and a triangle between polyA and Cas9 represents a loxP sequence, and a polyA represents an SV40 polyA sequence.

To make use of the self-targeting guide RNAs having low cytotoxicity and high stability, 11 of the 20 guide RNAs screened in Example 14 were cloned into a region of the stgRNA in the FLEx DNA library to prepare a library.

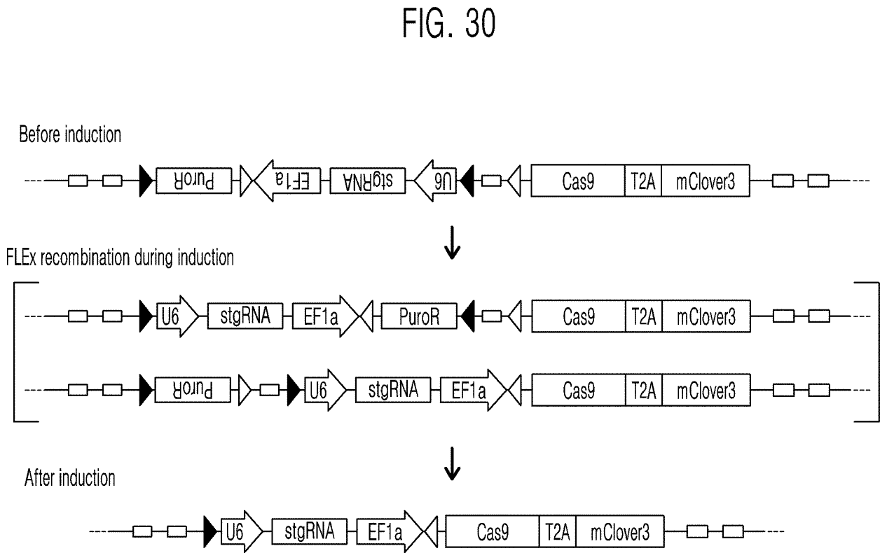

When a Cre protein is activated in a FLEx DNA clock library vector, a FLEx switch is operated by Cre-dependent recombination, thereby inducing the expression of the Cas9 nuclease whose activity had been turned off. When the expression of the Cas9 nuclease is initiated, formation of indels is induced in the sequence of the self-targeting guide RNA, thereby measuring the frequency of the indels formed in the sequence of the self-targeting guide RNA in the library to predict time (FIG. 30). When one lox2272 pair and one loxP pair are recombined by the Cre protein, Cas9 and mClover3 fluorescent proteins are expressed by an EF1a promoter regardless of order, and the indels are continuously formed in a region of the stgRNA.

As such, when the vector is designed to adjust the expression of the Cre protein by a promoter which is responsive to specific biological events, it is possible to measure the time at which the specific biological events occur.

### Example 16: Establishment of Cell Line Having Responsiveness to Specific Biological Events

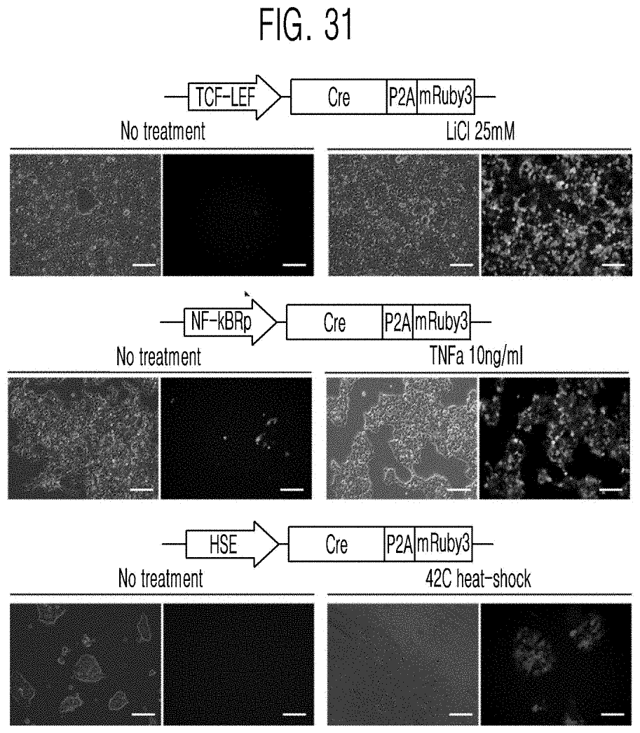

To determine temporal information on a variety of other various biological phenomena without any limitation on use of the Cas9-knockin cells or ciCas9-knockin cells, a lentiviral vector containing various synthetic promoters whose transcription is induced by certain stimuli was constructed. Lentiviral vectors for a TCF-LEF synthetic promoter responding to the Wnt signal transduction (Tang W et al., Science (2018)), an NF-kBR synthetic promoter activated by inflammatory responses (Perli S D et al., Science (2016)), and an HSE synthetic promoter responding to the heat induction (Ortner V et al., Cell Stress and Chaperones (2015)) were designed. Each of the lentiviral vectors was cloned to encode base sequences of a Cre protein and an mRuby3 fluorescent protein under the control of each of the corresponding synthetic promoters (FIG. 31).

Lentiviruses that express the Cre protein in response to the above-described three biological events were manufactured, and HEK293T cells were then infected with each of the lentiviruses, thereby establishing monoclonal cell lines. Such cell lines were subjected to each of Wnt transduction (treated with 25 mM LiCl), an inflammatory response (treated with 10 ng/mL hTNFa), and heat induction (incubated under 42° C. heat-shock conditions). As a result, it was confirmed that the mRuby3 fluorescent protein was expressed in all of the monoclonal cell lines.

### Example 17: Verification of DNA Clock System Operating in Response to Wnt Signal Transduction

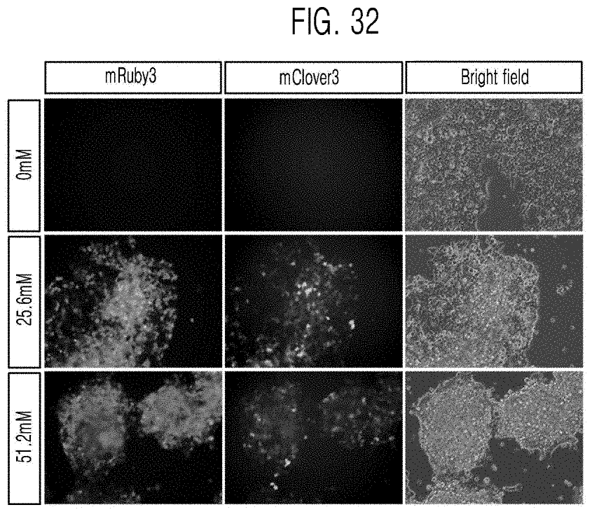

Among the established cell lines having responsiveness to the biological events, the FLEx DNA clock library was introduced into a cell line responding to the Wnt signal transduction to establish a DNA clock system operating in response to the Wnt signal transduction. The FLEx DNA clock library vector and the SB transposase vector were simultaneously transfected into the Wnt-responsive cell line, and the cell line was selectively incubated in a selective medium with puromycin. Then, the selected cell line was treated with LiCl to induce Cre-dependent recombination in the FLEx DNA clock library (FIG. 32).

The FLEx DNA clock library was introduced into a HEK293T monoclonal cell line responding to the Wnt signal transduction, and the Cre protein and the mRuby3 fluorescent protein were expressed by Wnt (treated with 25.6 mM, 51.2 mM LiCl). It was confirmed that the expression of Cas9 nuclease and mClover3 was induced by the Cre protein, indicating that the DNA clock system capable of measuring a time point at which the Wnt signal transduction occurred was operated.

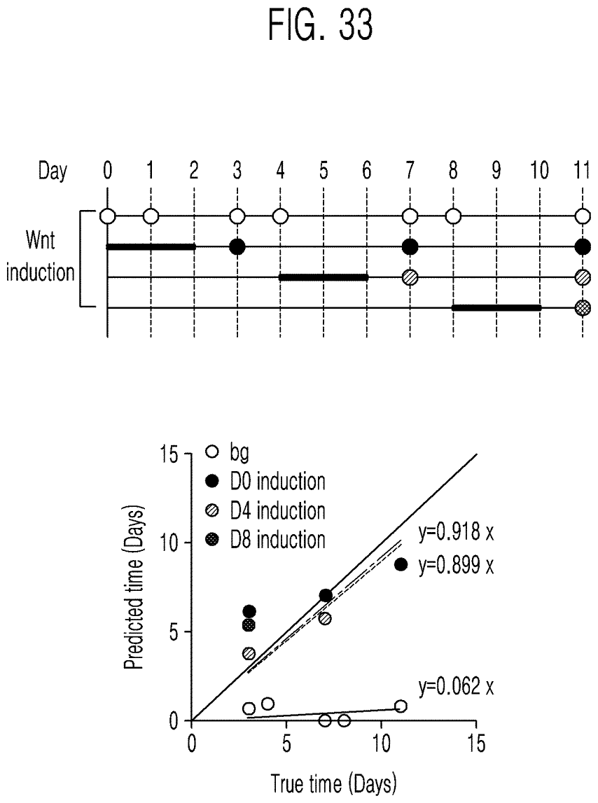

A Wnt signal was induced with LiCl at various start time points (days 0, 4, and 8) for 2 days in the Wnt-responsiveFLEx DNA clock library cells, and cells were collected at each of the indicated time points to analyze indel frequencies of the 11 self-targeting guide RNAs (FIG. 33).

There were two replicate groups per sample, and the half-lives of the self-targeting guide RNAs included in the library were estimated using the data on indel frequencies in one replicate group. The corresponding samples were taken at the time points indicated by circles in FIG. 33. In this case, empty circles represent control groups (bg) in which the Wnt signal was not induced, and circles indicated by colors or patterns represent samples in which the Wnt signal was induced for a period of time corresponding to the thick line. The time prediction values obtained using the data on indel frequencies of each sample are shown in the right graph of FIG. 33. As can be seen in the graph, it was confirmed that the time which had elapsed from the start time point at which each of the different Wnt signals were induced was well predicted when the time of another replicate group was predicted from the estimated half-lives. Therefore, it was confirmed that the FLEx DNA clock system capable of measuring a time point at which the certain biological phenomena were induced operated well.

[Experimental Materials]

The materials and sources used in Examples of the present invention are listed in the following Table 2.

TABLE 2

| REAGENT or RESOURCE | SOURCE | IDENTIFIER |
|---|---|---|
| Antibodies | | |
| Anti-CRISPR-Cas9 antibody [7A9- | Abcam | Cat# ab191468 |
| 3A3] | | |
| β-Actin Antibody (C4) | Santa Cruz | Cat# sc-47778 |
| | Biotechnology | |
| Bacteria and Viruses | | |
| One Shot Stbl3 Chemically | Thermo Fisher | Cat# C737303 |
| Competent E. coli | | |
| Subcloning Efficiency ™ DH5α ™ | Thermo Fisher | Cat# 18265017 |
| Competent Cells | | |
| Endura ™ ElectroCompetent Cells | Lucigen | Cat# 60242-2 |
| Compounds, Peptides and | | |
| Recombinant Proteins | | |
| BsmBI Restriction Enzyme | Enzynomics | Cat# R075L |
| Alkaline Phosphatase, Calf | NEB | Cat# M0290L |
| Intestinal (CIP) | | |
| NEBuilder ® HiFi DNA Assembly | NEB | Cat# E2621L |
| Master Mix | | |
| Phusion ® High-Fidelity DNA | NEB | Cat# M0530L |
| Polymerase | | |
| 2X Taq PCR Smart mix | Solgent | Cat# STD02-M50h |
| Fetal Bovine Serum (FBS) | Thermo Fisher | Cat# 16000-044 |
| DMEM | Thermo Fisher | Cat# 11995-065 |
| Penicillin-Streptomycin (10,000 | Thermo Fisher | Cat# 15140-122 |
| U/mL) | | |
| Lipofectamine 2000 transfection | Thermo Fisher | Cat# 11668-019 |
| Reagent | | |
| A-1155463 | BioVision | Cat# B1821 |
| Hygromycin B Gold ™ | InvivoGen | Cat# ant-hg-5 |
| Puromycin Dihydrochloride | Thermo Fisher | Cat# A1113803 |
| Zeocin ™ | InvivoGen | Cat# ant-m-1p |
| Protease Inhibitor Cocktail | Merck | Cat# P8340 |
| Assay | | |
| MEGAquick-spin ™ Total | iNtRON Biotechnology | Cat# 17290 |
| Fragment DNA Purification Kit | | |
| Wizard ™ Genomic DNA | Promega | Cat# A1620 |
| Purification Kit | | |
| Data | | |
| Deep Sequencing Data | NCBI | |
| Experiment Cell Lines | | |
| Flp-In ™ T-REx ™ Cell Line | Thermo Fisher | Cat# R780-07 |
| HEK293T Cells | ATCC | Cat# CRL-1573 |
| Cas9-E2A-mRFP Knockin Cell | This paper | N/A |
| Line | | |
| ciCas9-E2A-mRFP Knockin Cell | This paper | N/A |
| Line | | |
| Model Mouse (Organisms/Strains) | | |
| Mouse (NOG): NOD.Cg-Prkdc^{scid} | Central Institute for | N/A |
| Il2rg^{tm1Sug}/JicTac | Experimental Animals | |
| Oligonucleotide | | |
| All oligonucleotide pools used in | TwistBioscience | N/A |
| library construction | | |
| Primers used for library | This paper | N/A |
| construction (SEQ ID NOs: 1 to 3) | | |
| Primers used for deep sequencing | This paper | N/A |
| preparation (SEQ ID NOs: 5 to 20) | | |
| Primers used for stgRNA | This paper | N/A |
| expression quantification (SEQ ID | | |
| NOs: 21 to 24) | | |
| Recombinant DNA | | |
| Lenti_gRNA-Puro Plasmid | Addgene | Cat# 84752 |
| Lenti_stgRNA-Puro Plasmid | This paper | N/A |
| pRGEN-Cas9-CMV/T7-Puro-RFP | Toolgen | Cat# TGEN_0P1 |
| ciCas9_pcDNA5 | Addgene | Cat +190100550 |
| pcDNA ™5/FRT Expression Vector | Thermo Fisher | Cat# V6010-20 |
| pOG44 Expression Vector | Thermo Fisher | Cat# V6005-20 |
| pcDNA ™5/FRT/CMV_promoter- | This paper | N/A |
| Cas9-E2A-mRFP | | |
| pcDNA ™5/FRT/CMV_promoter- | This paper | N/A |
| ciCas9-E2A-mRFP | | |
| psPAX2 | Addgene | Cat# 12260 |
| pMD2.G | Addgene | Cat# 12259 |
| Software and Algorithms | | |
| EMBOSS | Rice, Longden, and | emboss. sourceforge.net |
| | Bleasby, 2000 | |
| R | R Core Team, 2018 | https://www.r-proj ect. org/ |
| doSNOW | Microsoft Corporation | https://crans.r- |
| | and Stephen Weston, | project.org/web/packages/doSNOW |
| | 2017 | /index.html |
| Indel searcher, model comparison, | This paper and | https://github.com/hkimlab/Supple |
| t₀ and half-life calculation | available on GitHub | mentalCodes |
| algorithms | | |
| Others | | |
| MicroPulser ™ Electroporator | Bio-Rad | Cat# 1652100 |
| QIAGEN Plasmid Maxi Kit | QIAGEN | Cat# 12165 |
| Millex-GV Syringe Filter Unit, | Merck | Cat# SLGV033RS |
| 0.22 μm, PVDF, 33 mm, gamma | | |
| sterilized | | |
| 48-well PS scaffold | 3D Biotek | Cat# PS152048-16 |

[Experimental Methods]

1. Construction of Vector

The backbone of a lentiviral plasmid for constructing Libraries 1 and 2 was constructed by modifying an sgRNA scaffold into a stgRNA scaffold through the site-specific mutagenesis from a Lenti_gRNA-Puro plasmid (Addgene; #84752). Specifically, the site-specific mutagenesis was performed by replacing positions U23 and U24 with guanine and replacing positions A48 and A49 with cytosine (Perli et al., 2016). The constructed vector was transformed into an E. coli strain Stbl3 (Thermo Fisher, Waltham, Mass.), and then screened in the presence of 100 μg/mL ampicillin.

To construct an integrated vector for preparing Cas9- and ciCas9-knockin cells, each of pRGEN-Cas9-CMV/T7-Puro-RFP (Toolgen, Seoul, Korea) and ciCas9pcDNA5 (Addgene; #100550) (Rose et al., 2017) cassettes was subcloned into a pcDNA™5/FRT expression vector (Thermo Fisher, Waltham, Mass.), and pcDNA™5/FRT/CMV_promoter-Cas9-E2A-mRFP and pcDNA™5/FRT/CMV_promoter-ciCas9-E2A-mRFP vectors were then constructed (FIGS. 1 and 16A). Each of the vectors was transformed into an E. coli strain DH5α (Thermo Fisher), and then screened in the presence of 100 μg/mL ampicillin.

2. Preparation of Cas9- or ciCas9-Expressing Cells

An Flp-In™ T-REx™ cell line (Thermo Fisher) was stored in a DMEM medium supplemented with 10% FBS (Gibco, Waltham Mass.). The cells were transfected with an Flp recombinant vector (pOG44 Expression Vector; Thermo Fisher) or a knockin vector including a ciCas9-E2A-mRFP sequence according to the instructions. After 48 hours, the Cas9- or ciCas9-knockin cells were cultured in the presence of 100 μg/mL hygromycin B Gold (InvivoGen, Pak Shek Kok, Hong Kong) for a week, and then screened. Each colony was picked using a pipette while observing the colonies using a phase-contrast microscope. Living cell colonies uniformly expressing mRFP were screened under a fluorescence microscope, and cultured in the presence of 20 μg/mL hygromycin B Gold. Then, each cell line was stored in a freezer. The frozen cell line was used in all the experiments in this specification after the cell line was thawed and cultured in a medium including 20 μg/mL hygromycin B Gold.

3. Western Blot

The entire cell eluate of Cas9-E2A-mRFP-knockin cells was prepared using a cell lysis buffer (50 mM Tris-HCl, pH 7.5, 1% Triton X-100, 150 mM NaCl, 0.1% sodium dodecyl sulfate, and 1% sodium deoxycholate) including a mixture of protease inhibitors (Merck, Darmstadt, Germany). The eluate was centrifuged at 13,000×g and 4° C. for 20 minutes to obtain a supernatant protein extract, which was then stored at −80° C. until use. A total of 30 μg of proteins per sample was loaded onto an 8% acrylamide gel, and then electrophoresed along a nitrocellulose membrane. The membrane was reacted overnight with a primary antibody (1:1,000) (Abcam, Cambridge, UK) or β-actin (1:1,000) (Santa Cruz Biotechnology, Dallas, Tex.) against an anti-CRISPR-Cas9 antibody at 4° C. The Western blot results were obtained using ImageQuant LAS 4000 (GE Healthcare, Velizy-Villacoublay, France) (FIG. 2A).

4. Design of Oligonucleotide Pool

To construct Library 1, 23,940 oligonucleotides having a total length of 138 nt (FIG. 3), each of which includes a 23-nt 5′-constant region sequence, a 15-nt barcode sequence, a 50-nt extension sequence, a 20-nt guide sequence, a 3-nt PAM sequence, and a 27-nt 3′-constant region sequence, were customized (TwistBioscience, San Francisco, Calif.). Thereafter, a target sequence used for deep sequencing analysis consisted of a 20-nt guide sequence and a 3-nt PAM sequence. The barcode sequence includes any sequence other than a 2-nt or more mononucleotide repeat sequence. To generate the 50-nt extension sequence, first, any two sequences having no 2-nt or more mononucleotide repeat sequence were generated, and the two sequences were then combined in an arbitrary manner. Among the 23,940 stgRNA-encoding sequences, 14,000 guide sequences were optionally designed to have GC contents of 40% or more and 60% or less without including the mononucleotide repeat sequence, and 9,800 guide sequences were optionally selected under the conditions in which the mononucleotide sequence had a length of 10 nt or less. The remaining 140 sequences was prepared by combining 10 different barcode and extension sequence sets with the previously used stgRNA-encoding sequence (Kaihor et al., 2016; Perli et al., 2016). Because four of the stgRNA-encoding sequences reported in previous articles includes a 10-nt or 20-nt extension sequence, the extension sequence was used as an additional extension sequence. When the above-described extension sequence had a length of 20-nt bases, the 5′- and 3′-constant region sequences were reduced by 20 nt and 22 nt, respectively.

To construct Library 2, 2,000 stgRNA-encoding sequences were selected from the Library 1 according to the of indel frequency ranking measured for the replicate group of Library 1 on day 3. Sequences having a minimum sequencing read depth of 50 and exhibiting a background indel frequency of 5% or less were selected as the target sequence. The top 1,800 sequences and the bottom 200 sequences were selected. A 20-nt guide sequence was replaced with a stgRNA-encoding sequence, in sequences ranked 1,201 to 1,800, to correspond to the guide sequence having high activity for SpCas9.

Target sequences for constructing Library 3 were selected from several thousands of target sequences according to an indel-generating activity profile obtained by testing levels of SpCas9 and sgRNA activities. In Library 3, the guide RNA-encoding sequences were more associated with a general sgRNA scaffold, compared to the stgRNA scaffold. To enhance a ratio of the guide RNAs having excellent activity in Library 3, the replicate groups (up to 10) of guide sequences having good activities were combined with a uniquely defined barcode sequence. To synthesize an array of Library 3, a 1,993 oligonucleotide pool having a total length of 150 nt, each of which includes a 20-nt 5′-constant region, a 20-nt guide sequence, a 11-nt first BsmBI restriction site, a 20-nt barcode 1 sequence, a 11-nt second BsmBI restriction site, a 15-nt barcode 2 sequence, a 3-nt arbitrary sequence (without mononucleotide repeats ≥2 nt), a corresponding target sequence including a 30-nt PAM sequence, and a 20-nt 3′-constant region sequence, were customized (TwistBioscience, San Francisco, Calif.).

5. Preparation of Plasmid Library

A backbone plasmid used in Libraries 1 and 2 was made linear by reacting with a BsmBI restriction enzyme (Enzynomics, Daejeon, Korea) at 55° C. for 3 hours. After the reaction with the restriction enzyme, the backbone was treated with 1 μL of calf intestinal alkaline phosphatase (NEB) at 37° C. for 30 minutes. An oligonucleotide was amplified by PCR using OligoAmp_pF1, pR1 (SEQ ID NOs: 1 and 2) primer sets and a Phusion polymerase (NEB), and the amplified product was gel-purified using a MEGAquick-Spin™ total fragment DNA purification kit (iNtRON Biotechnology, Seongnam, South Korea). The linear backbone plasmid and the purified PCR-amplified product of the oligonucleotide were ligated at 50° C. for 40 minutes using a NEBuiderHiFi DNA assembly kit (NEB). Thereafter, the ligated product was transformed into electrocompetent bacteria (Lucigen, Middleton, Wis.) using MicroPulser (Bio-Rad, Hercules, Calif.). The transformed bacteria were placed on an LB agar plate containing 50 μg/mL carbenicillin, and cultured at 37° C. for 16 hours. Thereafter, a plasmid was extracted from the cultured colonies using a Plasmid Maxiprep kit (Qiagen, Hilden, Germany). The plasmid library coverage was calculated according to the equation: (Total number of bacterial colonies)/(Total number of oligonucleotides in library). The final coverages of Libraries 1 and 2 were 3.83× and 20.6×, respectively.

In particular, Library 3 was established using a 2-step cloning method including a restriction enzyme digestion and ligation step and a Gibson assembly step. Such a multi-step method effectively prevents the uncoupling of a pair of the guide RNA and the target sequence in a PCR amplification process of an oligonucleotide pool. A specific method is as follows.

Step 1: Generating Initial Plasmid Library Including a Pair of Guide Sequence and Target Sequence

A Lenti-gRNA-Puro plasmid (Addgene; #84752) was made linear by reacting with a BsmBI enzyme (Enzynomics) at 55° C. for 6 hours. After the reaction with the restriction enzyme, a vector was treated with 1 μL of calf intestinal alkaline phosphatase (NEB) at 37° C. for 30 minutes. An oligonucleotide was amplified by PCR using an OligoAmp_pF1, pR2 (SEQ ID NOs: 1 and 3) primer set and a Phusion polymerase (NEB), and the amplified product was gel-purified, and the assembled with the linear backbone using a NEBuilder HiFi DNA assembly kit (NEB). The assembled product was purified, and then transformed into electrocompetent cells in the same manner as described above.

Primer sets used to amplify the oligonucleotides of the present invention are listed in the following Table 3.

TABLE 3

| | | SEQ ID |
|---|---|---|
| Name | Sequence | NO: |
| OligoAmp_pF1 | TTGAAAGTATTTCGATTTCTTGGCTTTA | 1 |
| | TATATCTTGTGGAAAGGACGAAACACC | |
| OligoAmp_pR1 | TTTCAAGTTGATAACGGACTAGCCTTAG | 2 |
| | GTTAACTTGCTATTTCTAGCTCTAAC | |
| OligoAmp_pR2 | GAGTAAGCTGACCGCTGAAGTACAAGTG | 3 |
| | GTAGAGTAGAGATCTAGTTACGCCAAGCT | |

Step 2: Knockin of sgRNA Scaffold

The initial plasmid library prepared in the step 1 was digested with BsmBI for 12 hours, and treated with 2 μL of calf intestinal alkaline phosphatase (NEB) at 37° C. for 30 minutes. The enzymatic reaction product was size-screened by means of 0.8% agarose gel electrophoresis, and then purified using a MEGAquick-spin total fragment DNA purification kit (iNtRON Biotechnology).

Separately, a synthetic knockin fragment including the sgRNA scaffold (SEQ ID NO: 4;

CGTCTCTGTTTTAGAGCTAGAAATAGCAAGTTAAAATAAGGCTAGTCCGTT
ATCAACTTGAAAAAGTGGCACCGAGTCGGTGCTTTTTTGGGAGACG)

was cloned into a TOPO vector (T-blunt vector; Solgent, Daejeon, South Korea). The sgRNA scaffold in this knockin fragment includes a poly-T sequence (bold) and a BsmBI restriction site (underlined).

Next, the TOPO vector including the knockin fragment was digested with BsmBI, and the 83-nt knockin fragment was purified in a 4% agarose gel. Four ligation reactions were performed using 90 ng of the purified knockin fragment and 200 ng of the initial plasmid library vector. The reaction mixture was reacted overnight at 16° C., and the reaction product was then thermally inactivated at 65° C. for 10 minutes, and purified in a column. The purified product was transformed into electrocompetent cells using the above-described method. As a result, the final plasmid library having a coverage of 3,990× for the initial number of oligonucleotides was obtained as Library 3. Colonies were harvested using a Plasmid Maxiprep kit (Qiagen), and a plasmid was extracted from the colonies.

6. Production of Lentiviruses

HEK293T cells (ATCC) were stored in a DMEM medium supplemented with 10% FBS and penicillin-streptomycin (pen-strep). For lentivirus production, a transfer plasmid including a desired gene, psPAX2 (Addgene; #12260), and pMD2.G (Addgene; #12259) were mixed in a 4:3:1 weight ratio to prepare a total of 60 μg of a plasmid mixture. Thereafter, the plasmid mixture was transferred to 70 to 80% confluent HEK293T cells using Lipofectamine 2000 (Invitrogen, Carlsbad, Calif.). At the time point of 24 hours after transfection, the medium was replaced with 20 mL of a growth medium. A supernatant including the viruses was harvested 72 hours after first transfection, filtered through a Millex-HV 0.45 μm low-protein-binding membrane (Merck, Darmstadt, Germany), and divided into aliquots, which were then stored at −80° C. in a freezer until immediately before use.

To measure the titer of the virus, the viral aliquots were serially diluted, and transduced into the HEK293T cells in the presence of 10 μg/mL polybrene. Non-transduced cells and the cells treated with the serially diluted viruses were cultured in the presence of 2 μg/mL puromycin. When almost all of the non-transduced cells were dead, the populations treated with the living viruses were counted to estimate the titer of the viruses.

7. Transduction of Libraries 1, 2 and 3

The Cas9-E2A-mRFP-knockin cells with the total cell number of 1.0×10⁸ (Library 1) or 4.8×10⁷ (Library 2) were seeded in a 150 mm tissue culture dish at a concentration of 1.0×10⁷ cells/dish (Library 1) or 1.2×10⁷ cells/dish (Library 2), and then cultured overnight. The cell batch was transduced with Lentiviral library 1 or 2 at MOI of 0.3 in the presence of 10 μg/mL polybrene, and then cultured for 24 hours. To remove non-transduced cells, the cells were cultured for 3 days in the presence of 2 μg/mL puromycin and 20 μg/mL hygromycin B Gold. To preserve the diversity of the cell library, the cell library was kept in the presence of 1 μg/ml puromycin and 20 μg/ml hygromycin B Gold so that the minimum number of the cells was 2.4×10⁷ cells. At each sampling time point (FIG. 5), at least 2.4×10⁷ (1,000× for Library 1, and 12,000× for Library 2) cells were harvested to isolate genomic DNA, and 80 μg of the genomic DNA corresponding to the 8.0×10⁶ cells (333× for Library 1, and 4,000× for Library 2) were used for deep sequencing analysis.

To transduce Lentiviral library 3, 6.0×10⁸ ciCas9-E2A-mRFP-knockin cells were seeded in five 150 mm tissue culture dishes at a concentration of 1.2×10⁷ cells/dish. 24 hours after transduction, 1 μg/mL puromycin was added to a culture medium. The cells were cultured for 24 hours, and then subcultured in 100 mm tissue culture dishes. The next day, the cells were treated with 10 μNI A-1155463 (BioVision, Milpitas, Calif.) to activate ciCas9. At each time point, at least 8.0×10⁶ cells (4,000×) were harvested, and 80 to 120 μg of genomic DNA corresponding to 0.8 to 1.2×10⁷ cells (4,000˜6,000×) was used for PCR amplification and deep sequencing.

8. Measurement of Background Indel Frequencies

To measure a background indel frequency of a target sequence, 2.4×10⁷ HEK293T cells were transduced with Lentiviral libraries 1 and 2, and genomic DNA was isolated after 3 days. Thereafter, an amount of 160 μg of genomic DNA corresponding to the 1.6×10⁷ cells (667× for Library 1; and 8,000× for Library 2) was amplified by PCR, and subjected to deep sequencing.

In the case of Library 2, ciCas9-E2A-mRFP-knockin cells were transduced with Lentivirus 3, and an amount of 240 μg of genomic DNA corresponding to the 2.4×10⁷ cells (12,000×) was analyzed after 3 days.

9. Deep Sequencing

Genomic DNA was extracted from a cell pellet using a Wizard Genomic DNA purification kit (Promega, Fitchburg, Wis.). Thereafter, the target sequence was amplified by PCR using a 2× Taq PCR Smart mix (Solgent).

To establish sufficient library diversity and improve the quality of deep sequencing results, two independent PCR reactions were performed. The first PCR reaction set for deep sequencing analysis was prepared using three pairs of forward and reverse primer sets (NGS1st_stgRNA_pF1,2,3 and pR1,2,3; SEQ ID NOs: 5 to 10) and ¾ of the extracted genomic DNA. The second PCR reaction set was prepared using ¼ of the remaining genomic DNA and one pair of primer sets (NGS1st_stgRNA_pF1r, pR1r; SEQ ID NOs: 11 and 12). For Libraries 1 and 2, a PCR reaction was performed on the samples taken at all the time points. For Library 3, three pairs of forward and reverse primer sets (NGS1st_sgRNA_pF1,2,3 and pR1,2,3; SEQ ID NOs: 13 to 18) were used to amplify the target sequences included in the genomic DNA of the samples taken at all the time points and subject the amplified target sequences to deep sequencing.

The first PCR-amplified product was combined in one pool, primarily purified using a MEGAquick-spin Total Fragment DNA Purification Kit (iNtRON Biotechnology), and then gel-purified using the same kit. The purified product was amplified by PCR using primers (NGS2nd_pF1, pR1; SEQ ID NOs: 19 and 20) containing Illumina adaptors. Thereafter, the amplified product was analyzed using HiSeq or MiniSeq (Illumina, San Diego, Calif.).

The primer sequences used in the PCR reaction for deep sequencing analysis according to the present invention are listed in the following Table 4.

TABLE 4

| | | | SEQ ID |
|---|---|---|---|
| Usage | Name | Sequence | NO: |
| 1^{st} PCR | NGS1st_stgRNA_pF1 | ACACTCTTTCCCTACACGACGCTCTTCCGATCTT | 5 |
| Reaction | | GGCTTTATATATCTTGTGGAAAGGACG | |
| (Libraries | NGS1st_stgRNA_pF2 | ACACTCTTTCCCTACACGACGCTCTTCCGATCTA | 6 |
| 1 and 2) | | TGGCTTTATATATCTTGTGGAAAGGACG | |
| | NGS1st_stgRNA_pF3 | ACACTCTTTCCCTACACGACGCTCTTCCGATCTC | 7 |
| | | CTGGCTTTATATATCTTGTGGAAAGGACG | |
| | NGS1st_stgRNA_pR1 | GTGACTGGAGTTCAGACGTGTGCTCTTCCGATCT | 8 |
| | | GCCTTAGGTTAACTTGCTATTTCTAGCTCTA | |
| | NGS1st_stgRNA_pR2 | GTGACTGGAGTTCAGACGTGTGCTCTTCCGATCT | 9 |
| | | TGCCTTAGGTTAACTTGCTATTTCTAGCTCTA | |
| | NGS1st_stgRNA_pR3 | GTGACTGGAGTTCAGACGTGTGCTCTTCCGATCT | 10 |
| | | ATGCCTTAGGTTAACTTGCTATTTCTAGCTCTA | |
| 1^{st} PCR | NGS1st_stgRNA_pF1r | GTGACTGGAGTTCAGACGTGTGCTCTTCCGATCT | 11 |
| Reaction | | TGGCTTTATATATCTTGTGGAAAGGACG | |
| (Reverse) | NGS1st_stgRNA_pR1r | ACACTCTTTCCCTACACGACGCTCTTCCGATCTG | 12 |
| (Libraries | | CCTTAGGTTAACTTGCTATTTCTAGCTCTA | |
| 1 and 2) | | | |
| 1^{st} PCR | NGS1st_sgRNA_pF1 | ACACTCTTTCCCTACACGACGCTCTTCCGATCTCT | 13 |
| Reaction | | TGAAAAAGTGGCACCGAGTCG | |
| (Library 3) | NGS1st_sgRNA_pF2 | ACACTCTTTCCCTACACGACGCTCTTCCGATCTTC | 14 |
| | | TTGAAAAAGTGGCACCGAGTCG | |
| | NGS1st_sgRNA_pF3 | ACACTCTTTCCCTACACGACGCTCTTCCGATCTC | 15 |
| | | GCTTGAAAAAGTGGCACCGAGTCG | |
| | NGS1st_sgRNA_pR1 | GTGACTGGAGTTCAGACGTGTGCTCTTCCGATCT | 16 |
| | | TTAAGTCGAGTAAGCTGACCGCTGAAG | |
| | NGS1st_sgRNA_pR2 | GTGACTGGAGTTCAGACGTGTGCTCTTCCGATCT | 17 |
| | | ATTAAGTCGAGTAAGCTGACCGCTGAAG | |
| | NGS1st_sgRNA_pR3 | GTGACTGGAGTTCAGACGTGTGCTCTTCCGATCT | 18 |
| | | TATTAAGTCGAGTAAGCTGACCGCTGAAG | |
| 2^{nd} PCR | NGS2nd_pF1 | AATGATACGGCGACCACCGAGATCTACACNNNN | 19 |
| Reaction | | NNNNACACTCTTTCCCTACACGAC | |
| | NGS2nd_pR1 | CAAGCAGAAGACGGCATACGAGA | 20 |
| | | GTGACTGGAGTTCAGACGTGT | |

10. Analysis of Indel Frequencies

The deep sequencing data was analyzed using a Python script developed in our laboratory (Kim et al., 2017). The target sequences in Libraries 1, 2, and 3 were determined using a 19-nt unique sequence including a barcode sequence (a 4-nt upstream sequence plus a 15-nt barcode sequence). An insertion or deletion site positioned at a region spanning four nucleotides upstream to four nucleotides downstream of the expected restriction site (3 nucleotides upstream from the PAM sequence) was considered to be modified by induction with SpCas9 (Kim et al., 2018; Kim et al., 2017).

To exclude the background indel frequencies produced during the oligo pool synthesis and PCR amplification processes, the observed indel frequencies were normalized with the background indel frequency.

$$Indel Frequency ( I F , \% ) = \frac{( Observed Indel Frequency ) - ( Background Indel Frequency )}{100 - ( Background Indel Frequency )} \times 100$$

For more accurate analysis, the guide sequences with a background indel frequency and an indel frequency of 5% or more were excluded, and an indel frequency of 0% or less was optionally set to 0%. In the present invention, other main values for time estimation were calculated as follows.

$$F = ( \% ) = 100 = I F$$

$$m u t k = \frac{I F}{100} \times k$$

$$n p s = a minimum value of \frac{k \times F}{100} and \frac{k \times I F}{100}$$

wherein F represents a relative frequency (%) of the copy number of an intact target sequence, k represents the total number of read counts per target sequence at each time point, and mutk represents the number of mutation read counts per target sequence at each time point.

In the same way, F, mutk, and nps values were calculated for the background data, and indicated by Back_F, Back_mutk, and Back_nps, respectively.

11. Comparison of Mathematical Models

To determine the optimal model, the fittings of linear, exponential, Gompertz, and logistic models were compared using R code.

$$Linear Mode \text{l:} {F}_{t} = 1 - β t , β > 0$$

$$Exponential Mode \text{l:} {F}_{t} = {e}^{- λ t} , λ > 0$$

$$or {F}_{t} = {e}^{- λ ( t - {t}_{0} )} , λ > 0 , {t}_{0} = 1.0 \; 21 days$$

$$Gompertz Mode \text{l:} {F}_{t} = {e}^{- {be}^{ct} + b} , b , c > 0$$

$$Logistic Mode \text{l:} {F}_{t} = \frac{1 + {e}^{- k {x}_{0}}}{1 + {e}^{k ( t - {x}_{0} )}} , k > 0 , - ∞ < {x}_{0} < ∞$$

As an estimator for estimating the relative fit of each of the models having different parameters, an Akaike information criterion (AIC) and a Bayesian information criterion (BIC) were used. All the data values obtained at each measurement time point in the replicate groups A to F were used without any limitation for calculation for unbiased comparison. Parameters were estimated according to the following equation using a least square method in which the residual sum of squares (RSS) becomes the minimum value.

$$\hat{θ} = {argmin}_{θ} {\sum }_{t = 1}^{n} {( \hat{{F}_{t}} - {F}_{t} )}^{2}$$

wherein θ represent a parameter set for each model. A closed-form solution was included in a linear model, but numerical algorithms were used to estimate the parameters in other models.

AIC and BIC values were calculated according to the following equation.

$$A I C = 2 p + n \times ( ln ( 2 π \times \frac{R S S}{n} ) + 1 )$$

$$B I C = ln ( n ) \times p + n \times ( ln ( 2 \times \frac{R S S}{n} ) + 1 )$$

wherein p represents the total number of parameters used in the mathematical model, and n represents the number of time points observed for each guide sequence. In the exponential model, a AAIC or ABIC value was calculated by subtracting AIC and BIC values of each target sequence from the values of the three other different models. For the exponential model, the latency period (t₀) was assumed to be 0 or 1.021.

12. Estimation of Latency Period

The half-life and latency period (t₀) associated with each of the guide sequences were estimated using the above-described R code. The most suitable half-life and latency period (t₀) (for minimizing the RSS) were estimated using the individual t₀ values determined for the replicate groups A to F at all the time points. The target sequences having an intact target frequency of 85% or higher at all the analysis time points were excluded from the analysis. Also, all the data satisfying the requirement: 2%<f_{t,i}<95% was used for calculation.

To remove outliers, the top 5% t₀ values and the bottom 5% t₀ values were excluded, and the nps-weighted mean was calculated as the final t₀ value. A total of 39,138 individual t₀ values were used, and the finally calculated t₀ value was 1.021 days.

After the t₀ value was determined, the t₀ value was corrected to 1.021 days to re-calculate the half-lives of the target sequences from all the replicate groups of Libraries 1 and 2. Unless indicated otherwise, all the analyses using Libraries 1 and 2 were performed with the t₀ value set to 1.021 days. However, in the case of Library 3, the analysis was performed with the t₀ value set to 0 hour because the chemically-inducible Cas9 was immediately activated.

13. Time Estimation

For accurate estimation of the time (\hat{t}ₜ), the half-life of each of the target sequences was first calculated using the R code for minimizing the RSS of the exponential model in a state in which the t₀ value was set to 1.021 days. Thereafter, the final half-life of each of the target sequence was calculated as the weighted mean value of each of the half-lives from all of the replicate groups. The final half-life was weighted according to the sum of nps values at all the observation time points for each of the target sequence.

Next, the calculated f_{t,i} value was applied to the inverse function

$$( {\hat{t}}_{t , i} = - \frac{ln ( {f}_{t} )}{λ} + {t}_{0} )$$

of an exponential model equation to calculate a \hat{t}_{t,i} value of each of the target sequences at a given time point t. In this case, the \hat{t}_{t,i} value was calculated with the t₀ values set to 1.021 days in the case of Libraries 1 and 2 and 0 day in the case of Library 3. Then, the \hat{t}_{t,i} values included in a quartile range (spanning from the 25th to 75th percentiles) were selected from a pool of the \hat{t}_{t,i} values at each time point, and the nps-weighted mean (\hat{t}ₜ) of the selected \hat{t}_{t,i} values was calculated as follows.

$${\hat{t}}_{t} = \frac{{\sum }_{i = j}^{n} n p {s}_{t , i} \times {\hat{t}}_{t , i}}{{\sum }_{i = j}^{n} n p {s}_{t , i}}$$

wherein \hat{t}_{t,j} and \hat{t}_{t,n} represent \hat{t}_{t,i} values of the 25^{th} percentile and 75^{th} percentile at each given time point t, respectively. The error of the estimated \hat{t}ₜ value at a true time point was calculated as follows.

$$R A {E}_{t} ( Relative absolute error , \% ) = \frac{\vert {\hat{t}}_{t} - {t}_{true} \vert}{{t}_{t r u e}} \times 100$$

The mean values (mean RAE and MRAE) of RAEₜ at all the time points were used as the parameters for the accuracy of time estimation.

14. Sub-Sampling of Target Sequences

To check how much the number of the target sequences affected the accuracy of time estimation, sub-samples were randomly extracted from Libraries 1 and 2 (FIG. 15). The “RAND( )” function of MS Excel was used to determine the number of the target sequences and select library sub-samples having 10 different barcode sequences in each replicate group.

15. In Vivo Transplantation of Cas9-Knockin Cells Transduced with Library 2

In this example, all animal experiments were performed according to the regulations of the Institutional Animal Care and Use Committee (IACUC) at the College of Medicine of Yonsei University.

First, Cas9-E2A-mRFP-knockin cells were transduced with Lentiviral library 2 at MOI of 0.5. 24 hours after transduction, the cells were cultured for 3 days in the presence of 2 μg/mL puromycin and 20 μg/mL hygromycin B Gold to remove non-transduced cells. Thereafter, the 1.0×10⁶ cells were seeded in a non-degradable polystyrene 48-well pore scaffold (3D Biotek, Bridgewater, N.J.) at a concentration of 1.0×10⁶ cells/scaffold, and then cultured for 24 hours in a culture medium. The cells seeded in the scaffold were subcutaneously injected into the back and other quadrants of a male NOG mouse (NOD/Shi-scid/IL-2Rγnull) at a concentration of 4 scaffolds/mouse. As the in vitro control group, some of the cells seeded in the scaffold were cultured in a culture medium. On days 8, 14, and 21 after the transduction of Library 2, the scaffold was harvested.

To isolated genomic DNA from the cells in the scaffold, the cells including each scaffold were placed in a 2 mL Eppendorf tube containing 2 mL of a DNA elution buffer (Wizard Genomic DNA purification kit; Promega), and then cultured overnight while stirring. The genomic DNA was isolated from the cell eluate, and then subjected to deep sequencing using the above-described method.

The first PCR reaction for deep sequencing was performed using 48 μg of genomic DNA (2,400×) and three pairs of primer sets (NGS1st_stgRNA_pF1,2,3 and pR1,2,3; SEQ ID NOs: 5 to 10).

1. A method for measuring time which has elapsed from a predetermined time point in cells, comprising:
(a) transducing a composition for editing target genes into cells, followed by culturing of the cells;
(b) harvesting some of the cultured cells at any time point (t) which has elapsed from a predetermined time point, followed by sequencing of a target sequence from the genomic DNA of the cells;
(c) measuring an indel frequency (IF) of the target sequence; and
(d) calculating any time point using the following equation:
F=1−IF=e^{-λ(t-t}^{₀}⁾(t≥0,t₀≥0)
(wherein F represents a relative frequency (ratio) of the copy number of an intact target sequence in the total copy number of the target sequence at any time point, IF represents an indel frequency of the target sequence measured at any time point, λ is a positive constant that represents an indel generation rate of the target sequence per unit time, and to is the latent time taken to express a transgene transduced into cells).

2. The method of claim 1, further comprising estimating a lambda constant (λ), which includes the following steps prior to the step (b):
(i) harvesting some of the cultured cells at predetermined time point (t*);
(ii) sequencing the target sequence from the genomic DNA of the cells;
(iii) measuring a frequency (F) of the copy number of an intact sequence in the total copy number of the target sequence; and
calculating an indel generation rate constant (λ) of the target sequence per unit time for the given target sequence using the following equation:
F=e^{-λt*}(t*≥0)
(wherein F represents a frequency of the copy number of an intact target sequence in the total copy number of the target sequence, A represents a positive constant, and t is a positive constant that represents a predetermined time point).

3. The method of claim 1, wherein the composition for editing target genes in the step (a) comprises a guide RNA, a target base sequence targeted by the guide RNA, and an RNA-guided nuclease.

4. The method of claim 1, wherein the composition for editing target genes in the step (a) comprises a self-targeting guide RNA (stgRNA), which comprises a guide RNA and a target sequence targeted by the guide RNA, and an RNA-guided nuclease.

5. The method of claim 1, wherein the step (a) comprises:
(i) preparing a cell line in which a sequence encoding the RNA-guided nuclease is inserted (knockin);
(ii) manufacturing a vector, which comprises a base sequence encoding the guide RNA and a target sequence targeted by the guide RNA;
(iii) transducing the vector into the cell line to prepare transduced cells; and
(iv) culturing the transduced cells.

6. The method of claim 3, wherein the activity of the RNA-guided nuclease is induced by a Cas9 protein, a Cpf1 protein, or chemicals.

7. The method of claim 6, wherein the Cas9 protein is derived from one or more selected from the group consisting of the genera Streptococcus, Neisseria, Pasteurella, Francisella, and Campylobacter.

8. The method of claim 6, wherein the Cpf1 protein is derived from one or more selected from the group consisting of Candidatus Paceibacter, Candidatus Methanoplasma, and the genus Lachnospira, Butyrivibrio, Peregrinibacteria, Acidominococcus, Porphyromonas, Prevotella, Francisella or Eubacterium.

9. The method of claim 5, wherein the base sequence encoding the guide RNA and the target sequence targeted by the guide RNA comprises two or more different sequences.

10. The method of claim 5, wherein the base sequence encoding the guide RNA and the target sequence targeted by the guide RNA are base sequences encoding a self-targeting guide RNA (stgRNA).

11. The method of claim 10, wherein the self-targeting guide RNA comprises two or more different sequences.

12. The method of claim 5, wherein the vector is a viral vector.

13. The method of claim 12, wherein the vector comprises one or more selected from the group consisting of a lentiviral vector or a retroviral vector, and a plasmid vector.

14. The method of claim 5, further comprising:
constructing a vector library comprising two or more vectors, which comprise base sequences encoding two or more guide RNAs and target sequences targeted by the respective guide RNAs; and
constructing a cell library comprising two or more cells in which the vectors are transduced into different cell lines.

15. The method of claim 1, wherein the sequencing of the target sequence is performed using deep sequencing.

16. A system for measuring time in cells comprising:
an intracellular indel generation unit comprising a composition for editing target genes;
an intracellular indel frequency measurement unit for sequencing of the target genes; and
a time prediction unit for calculating the lapse of time at any time point from a predetermined time point using the measured indel frequency.

17. The system of claim 16, wherein the composition for editing target genes comprises a guide RNA, a target base sequence targeted by the guide RNA, and an RNA-guided nuclease.

18. The system of claim 17, wherein the guide RNA and the target base sequence targeted by the guide RNA are base sequences encoding a self-targeting guide RNA.

19. The system of claim 16, wherein the sequencing at the indel frequency measurement unit is performed using deep sequencing.

20. The system of claim 16, wherein the time prediction unit calculates any time point using the following equation:
F=1−IF=e^{-λ(t-t}^{₀}⁾(t≥0,t₀≥0)
(wherein F represents a relative frequency (ratio) of the copy number of an intact target sequence in the total copy number of the target sequence at any time point, IF represents an indel frequency of the target sequence measured at any time point, λ is a positive constant that represents an indel generation rate of the target sequence per unit time, and to is the latent time taken to express a transgene transduced into cells).
